SEQUENCE LISTING (30 sequences)

SEQ ID NO: 1 - DNA, 15 bp, Homo sapiens
agaccccatctctaa

SEQ ID NO: 2 - DNA, 17 bp, Homo sapiens
gcctgggtgacagagca

SEQ ID NO: 3 - DNA, 13 bp, Homo sapiens
gacagagcaagac

SEQ ID NO: 4 - DNA, 16 bp, Homo sapiens
tctcggctcactgcaa

SEQ ID NO: 5 - DNA, 16 bp, Homo sapiens
tctcggctcactgcaa

SEQ ID NO: 6 - DNA, 16 bp, Homo sapiens
ggattacaggcgtgag

SEQ ID NO: 7 - DNA, 18 bp, Homo sapiens
cctgtaatcccagctact

SEQ ID NO: 8 - DNA, 16 bp, Homo sapiens
ggctggagtgcagtgg

SEQ ID NO: 9 - DNA, 18 bp, Homo sapiens
tgggtgacagagcaagac

SEQ ID NO: 10 - DNA, 14 bp, Homo sapiens
ctggagtgcagtgg

SEQ ID NO: 11 - DNA, 16 bp, Homo sapiens
ccactgcactccagcc

SEQ ID NO: 12 - DNA, 21 bp, Escherichia coli
gaatccccaggagcttacata

SEQ ID NO: 13 - DNA, 16 bp, Escherichia coli
cccaggagcttacata

SEQ ID NO: 14 - DNA, 20 bp, Escherichia coli
gtgaccggggtgagggcgtg

SEQ ID NO: 15 - DNA, 11 bp, Escherichia coli
agtgactgggg

SEQ ID NO: 16 - DNA, 18 bp, Saccharomyces cerevisiae
aagatgacgcaaatgatg

SEQ ID NO: 17 - DNA, 18 bp, Saccharomyces cerevisiae
gaagatgacgcaaatgat

SEQ ID NO: 18 - DNA, 34 bp, Artificial Sequence
  Description of Artificial Sequenceprobe for cytochrome B
aaactgcagcccctcagaatgatatttgtcctca

SEQ ID NO: 19 - DNA, 35 bp, Artificial Sequence
  Description of Artificial Sequenceprobe for cytochrome B
aaaaagcttccatccaacatctcagcatgatgaaa

SEQ ID NO: 20 - DNA, 12 bp, Homo sapiens
  Description of Artificial Sequencehuman cytochrome B
ccagacgcctca

SEQ ID NO: 21 - DNA, 12 bp, Homo sapiens
  human cytochrome B
accttcacgcca

SEQ ID NO: 22 - DNA, 11 bp, Unknown
  Description of Unknown Organismcommon probe to cytochrome B
tgccgagacgt

SEQ ID NO: 23 - DNA, 22 bp, Artificial Sequence
  Description of Artificial Sequenceamplification primer
ggagctgcagatgctgaccaac

SEQ ID NO: 24 - DNA, 20 bp, Artificial Sequence
  Description of Artificial Sequenceamplification primer
gctactggccgctgaagggc

SEQ ID NO: 25 - DNA, 20 bp, Artificial Sequence
  Description of Artificial Sequenceinterrogation primer
gctgaccatcaataaggaag

SEQ ID NO: 26 - DNA, 11 bp, Human immunodeficiency virus
  probe for ′colirep′ sequence from E. coli
agtgactgggg

SEQ ID NO: 27 - DNA, 12 bp, chicken
  Description of Artificial Sequencechicken cytochrome B
gcagacacatcc

SEQ ID NO: 28 - DNA, 12 bp, chicken
  chicken cytochrome B
ggaatctccacg

SEQ ID NO: 29 - DNA, 12 bp, cow
  bovine cytochrome B
acatacacgcaa

SEQ ID NO: 30 - DNA, 12 bp, dog
  canine cytochrome B
atatgcacgcaa

(19) United States
(12) Patent Application Publication
(10) Pub. No.: US 2003/0022163 A1
(43) Pub. Date: Jan. 30, 2003

(54) Detection of repeated nucleic acid sequences

(76) Inventors: Michelle N. Mandrekar, Oregon, WI (US); Allan Tereba, Fitchburg, WI (US); John William Shultz, Verona, WI (US)

Correspondence Address: WELSH & KATZ, LTD, 120 S RIVERSIDE PLAZA, 22ND FLOOR, CHICAGO, IL, 60606, US

(21) Appl. No.: 09/739,909

(22) Filed: Dec. 15, 2000

Related U.S. Application Data
(63) Continuation-in-part of application No. 09/383,316, filed on Aug. 25, 1999, now Pat. No. 6,391,551.
Application No. 09/383,316 is a continuation-in-part of application No. 09/358,972, filed on Jul. 21, 1999, now Pat. No. 6,235,480.

(51) Int. Cl. C12Q 1/68; C07H 21/04

(52) U.S. Cl. 435/6; 536/24.3

(57) ABSTRACT

Materials and methods are disclosed for the detection of a desired nucleic acid that contains multiple repeats of a predetermined nucleic acid target sequence, typically using nucleic acid hybridization methods. Nucleic acid hybrid detection methods can be used to qualitatively and quantitatively analyze for the presence of a desired nucleic acid. Applications of the disclosed materials and methods include the detection of nucleic acid without the need for nucleic acid amplification or isolation of a fragment containing nucleic acid target.

### CROSS-REFERENCE TO RELATED APPLICATIONS

This application is a continuation-in-part of U.S. Ser. No. 09/383,316 filed on Aug. 25, 1999, which is a continuation-in-part of allowed U.S. Ser. No. 09/358,972, filed on Jul. 21, 1999, the disclosures of all of which are incorporated in full herein by reference.

### BACKGROUND OF THE INVENTION

Methods to detect nucleic acids and to detect specific nucleic acids of interest provide a foundation upon which the large and rapidly growing field of molecular biology is built. There is constant need for alternative methods and products. The reasons for selecting one method over another are varied, and include a desire to avoid radioactive materials, the lack of a license to use a technique, the cost or availability of reagents or equipment, the desire to minimize the time spent or the number of steps, the accuracy or sensitivity for a certain application, the ease of analysis, or the ability to automate the process.

The detection and/or quantification of specific biomolecules from biological samples (e.g. tissue, sputum, urine, blood, semen, saliva) has applications in forensic science, such as the identification and exclusion of criminal suspects and paternity testing as well as medical diagnostics.

Accurate measurement of human DNA in samples that may contain DNA from various other sources is a problem commonly encountered in the forensic community. This problem has been addressed using a variety of hybridization and amplification techniques, however, none of the techniques of the art have resulted in the development of a simple, rapid test that can accurately determine the amount of human DNA in a potentially mixed DNA sample, either qualitatively or quantitatively.

Hybridization methods to detect nucleic acids are dependent upon knowledge of the nucleic acid sequence. Many known nucleic acid detection techniques depend upon specific nucleic acid hybridization in which an oligonucleotide probe is hybridized or annealed to nucleic acid in the sample or on a blot, and the hybridized probes are detected.

A traditional type of process for the detection of hybridized nucleic acid uses labeled nucleic acid probes to hybridize to a nucleic acid sample. For example, in a Southern blot technique, a nucleic acid sample is separated in an agarose gel based on size and affixed to a membrane, denatured, and exposed to the labeled nucleic acid probe under hybridizing conditions. If the labeled nucleic acid probe forms a hybrid with the nucleic acid on the blot, the label is bound to the membrane. Probes used in Southern blots have been labeled with radioactivity, fluorescent dyes, digoxygenin, horseradish peroxidase, alkaline phosphatase and acridinium esters.

Another type of process for the detection of hybridized nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target sequence. If the nucleic acid primers do not hybridize to the sample, then there is no corresponding amplified PCR product. In this case, the PCR primer acts as a hybridization probe. PCR-based methods are of limited use for the detection of nucleic acid of unknown sequence.

In a PCR method, the amplified nucleic acid product may be detected in a number of ways, e.g. incorporation of a labeled nucleotide into the amplified strand by using labeled primers. Primers used in PCR have been labeled with radioactivity, fluorescent dyes, digoxygenin, horseradish peroxidase, alkaline phosphatase, acridinium esters, biotin and jack bean urease. PCR products made with unlabeled primers may be detected in other ways, such as electrophoretic gel separation followed by dye-based visualization.

Fluorescence techniques are also known for the detection of nucleic acid hybrids. U.S. Pat. No. 5,691,146 describes the use of fluorescent hybridization probes that are fluorescence-quenched unless they are hybridized to the nucleic acid target sequence. U.S. Pat. No. 5,723,591 describes fluorescent hybridization probes that are fluorescence-quenched until hybridized to the nucleic acid target sequence, or until the probe is digested. Such techniques provide information about hybridization, and are of varying degrees of usefulness for the determination of single base variances in sequences. Some fluorescence techniques involve digestion of a nucleic acid hybrid in a 5′ to 3′ direction to release a fluorescent signal from proximity to a fluorescence quencher, for example, TaqMan® (Perkin Elmer; U.S. Pat. Nos. 5,691,146 and 5,876,930).

Enzymes having template-specific polymerase activity for which some 3′ to 5′ depolymerization activity has been reported include E. coli DNA Polymerase (Deutscher and Kornberg, J. Biol. Chem., 244(11):3019-28 (1969)), T7 DNA Polymerase, (Wong et al., Biochemistry, 30:526-37 (1991); Tabor and Richardson, J. Biol. Chem., 265: 8322-28 (1990)), E. coli RNA polymerase (Rozovskaya et al., Biochem. J., 224:645-50 (1994)), AMV and RLV reverse transcriptases (Srivastava and Modak, J. Biol. Chem., 255: 2000-4 (1980)), and HIV reverse transcriptase (Zinnen et al., J. Biol. Chem., 269:24195-202 (1994)). A template-dependent polymerase for which 3′ to 5′ exonuclease activity has been reported on a mismatched end of a DNA hybrid is phage 29 DNA polymerase (de Vega, M. et al. EMBO J., 15:1182-1192, 1996).

A variety of methodologies currently exist for detection of single nucleotide polymorphisms (SNPs) that are present in genomic DNA. SNPs are DNA point mutations or insertions/deletions that are present at measurable frequencies in the population. SNPs are the most common variations in the genome. SNPs occur at defined positions within genomes and can be used for gene mapping, defining population structure, and performing functional studies. SNPs are useful as markers because many known genetic diseases are caused by point mutations and insertions/deletions.

In rare cases where an SNP alters a fortuitous restriction enzyme recognition sequence, differential sensitivity of the amplified DNA to cleavage can be used for SNP detection. This technique requires that an appropriate restriction enzyme site be present or introduced in the appropriate sequence context for differential recognition by the restriction endonuclease. After amplification, the products are cleaved by the appropriate restriction endonuclease and products are analyzed by gel electrophoresis and subsequent staining. The throughput of analysis by this technique is limited because samples require processing, gel analysis, and significant interpretation of data before SNPs can be accurately determined.

Single strand conformational polymorphism (SSCP) is a second technique that can detect SNPs present in an amplified DNA segment (Hayashi, K. Genetic Analysis: Techniques and Applications, 9:73-79, 1992). In this method, the double stranded amplified product is denatured and then both strands are allowed to reanneal during electrophoresis in non-denaturing polyacrylamide gels. The separated strands assume a specific folded conformation based on intramolecular base pairing. The electrophoretic properties of each strand are dependent on the folded conformation. The presence of single nucleotide changes in the sequence can cause a detectable change in the conformation and electrophoretic migration of an amplified sample relative to wild type samples, allowing SNPs to be identified. In addition to the limited throughput possible by gel-based techniques, the design and interpretation of SSCP based experiments can be difficult. Multiplex analysis of several samples in the same SSCP reaction is extremely challenging. The sensitivity required in mutation detection and analysis has led most investigators to use radioactively labeled PCR products for this technique.

In the amplification refractory mutation system (ARMS, also known as allele specific PCR or ASPCR), two amplification reactions are used to determine if a SNP is present in a DNA sample (Newton et al. Nucl. Acids Res. 17:2503, 1989; Wu et al. Proc. Nat'l. Acad. Sci., U.S.A, 86:2757, 1989). Both amplification reactions contain a common primer for the amplification target of interest. The first reaction contains a second primer specific for the wild type product which will give rise to a PCR product if the wild type gene is present in the sample. The second PCR reaction contains a primer that has a single nucleotide change at or near the 3′ end that represents the base change that is present in the mutated form of the DNA. The second primer, in conjunction with the common primer, will only function in PCR if genomic DNA that contains the mutated form of genomic DNA is present. This technique requires duplicate amplification reactions to be performed and analyzed by gel electrophoresis to ascertain if a mutated form of a gene is present. In addition, the data must be manually interpreted.

Single base extension is a technique that allows the detection of SNPs by hybridizing a single strand DNA probe to a captured DNA target (Nikiforov, T. et al. Nucl. Acids Res., 22:4167-4175). Once hybridized, the single strand probe is extended by a single base with labeled dideoxynucleotides. The labeled, extended products are then detected using calorimetric or fluorescent methodologies.

A variety of technologies related to real-time (or kinetic) PCR have been adapted to perform SNP detection. Many of these systems are platform based, and require specialized equipment, complicated primer design, and expensive supporting materials for SNP detection. In contrast, the process of this invention has been designed as a modular technology that can use a variety of instruments that are suited to the throughput needs of the end-user. In addition, the coupling of luciferase detection sensitivity with standard oligonucleotide chemistry and well-established enzymology provides a flexible and open system architecture. Alternative analytical detection methods, such as mass spectroscopy, HPLC, and fluorescence detection methods can also be used in the process of this invention, providing additional assay flexibility.

SNP detection using real-time amplification relies on the ability to detect amplified segments of nucleic acid as they are during the amplification reaction. Three basic real-time SNP detection methodologies exist: (i) increased fluorescence of double strand DNA specific dye binding, (ii) decreased quenching of fluorescence during amplification, and (iii) increased fluorescence energy transfer during amplification (Wittwer, C. et al. Biotechniques, 22:130-138, 1997). All of these techniques are non-gel based and each strategy will be briefly discussed.

A variety of dyes are known to exhibit increased fluorescence in response to binding double stranded DNA. This property is utilized in conjunction with the amplification refractory mutation system described above to detect the presence of SNP. Production of wild type or mutation containing PCR products are continuously monitored by the increased fluorescence of dyes such as ethidium bromide or SYBER Green as they bind to the accumulating PCR product. Note that dye binding is not selective for the sequence of the PCR product, and high non-specific background can give rise to false signals with this technique.

A second SNP detection technology for real time PCR, known generally as exonuclease primers (TaqMan probes), utilizes the 5′ exonuclease activity of thermostable polymerases such as Taq to cleave dual-labeled probes present in the amplification reaction (Wittwer, C. et al, Biotechniques, 22:130-138, 1997; Holland, P. et al Proc. Nat'l. Acad. Sci., U.S.A, 88:7276-7280, 1991). While complementary to the PCR product, the probes used in this assay are distinct from the PCR primer and are dually-labeled with both a molecule capable of fluorescence and a molecule capable of quenching fluorescence. When the probes are intact, intramolecular quenching of the fluorescent signal within the DNA probe leads to little signal. When the fluorescent molecule is liberated by the exonuclease activity of Taq during amplification, the quenching is greatly reduced leading to increased fluorescent signal.

An additional form of real-time PCR also capitalizes on the intramolecular quenching of a fluorescent molecule by use of a tethered quenching moiety. The molecular beacon technology utilizes hairpin-shaped molecules with an internally-quenched fluorophore whose fluorescence is restored by binding to a DNA target of interest (Kramer, R. et al., Nature Biotechnol., 14:303-308, 1996). Increased binding of the molecular beacon probe to the accumulating PCR product can be used to specifically detect SNPs present in genomic DNA.

A final general fluorescent detection strategy used for detection of SNPs in real time utilizes synthetic DNA segments known as hybridization probes in conjunction with a process known as fluorescence resonance energy transfer (FRET) (Wittwer, C. et al. Biotechniques, 22:130-138, 1997; Bernard, P. et al., Am. J. Pathol., 153:1055-1061, 1998). This technique relies on the independent binding of labeled DNA probes on the target sequence. The close approximation of the two probes on the target sequence increases resonance energy transfer from one probe to the other, leading to a unique fluorescence signal. Mismatches caused by SNPs that disrupt the binding of either of the probes can be used to detect mutant sequences present in a DNA sample.

In summary, there is a need for alternative methods for the detection of nucleic acid hybrids. There is a great demand for such methods to determine the presence or absence of nucleic acid sequences that differ slightly from sequences that might otherwise be present. There is a great demand for methods to determine the presence or absence of sequences unique to a particular source in a sample. There is also a great demand for nucleic acid detection and quantification methods that are more highly sensitive than the known methods, highly reproducible and automatable.

It would be beneficial if another method were available for detecting the presence of a sought-after, predetermined nucleic acid target sequence. It would also be beneficial if such a method were operable using a sample size of the microgram to picogram scale. It would further be beneficial if such a detection method were capable of providing multiple analyses in a single assay (multiplex assays). The disclosure that follows provides such methods.

### BRIEF SUMMARY OF THE INVENTION

The present disclosure describes several nucleic acid sequences that are useful for the source-specific detection, and optionally quantification, of nucleic acid in a sample in an embodiment of the present invention. When a nucleic acid sequence is present, the present invention provides a highly sensitive means for quantitative detection of that nucleic acid sequence.

The presently disclosed nucleic acid probe sequences have advantage over sequences disclosed in the art in their observed utility for source-selectivity, and also because they are probes to nucleic acid target sequences that are present at high enough copy number in a raw sample that it is possible to detect and quantify the presence of a nucleic acid without the need to undergo a nucleic acid target sequence enrichment step prior to a nucleic acid hybrid detection step.

Nucleic acid target sequence enrichment steps are well known in the art and include, for example, PCR amplification of nucleic acid containing the target sequence, and cutting nucleic acid in a raw sample (e.g. with a restriction endonuclease) and separating nucleic acid containing the target from nucleic acid that does not contain the target sequence. As used here, “raw sample” refers to a sample that would benefit from further sample preparation steps, for example nucleic acid purification, prior to a source-specific nucleic acid analysis according to the present invention.

The repetitive sequences of the present invention are particularly useful for source-specific nucleic acid detection. The selected repetitive sequences are present in high copy number only in the source of interest. Preferably, the analytical output is at least two-fold, and more preferably five-fold and even more preferably ten-fold, over the sample controls including nucleic acid fromorganisms that are not being detected.

Situations where source-specific nucleic acid detection is useful include determining the source of an unknown nucleic acid sample or detection and quantification of the presence or identity of a contaminant. For example, a forensic laboratory might use source-specific nucleic acid detection to ascertain whether blood came from a human or a pet. A food science laboratory might use source-specific nucleic acid detection to ascertain whether a beef or chicken sample is contaminated with E. coli.

Although one of the advantages of the methods of the present invention using highly repetitive nucleic acid target sequence to detect and quantify the presence of a nucleic acid is that sensitivity enhancement via amplification of the target sequence is not necessary, there may be situations where amplification is desirable, depending on the desired sensitivity of the assay.

The method of this embodiment of the present invention uses highly repetitive sequences as targets for sensitive nucleic acid detection. Novel probes are provided that form nucleic acid hybrids with their respective nucleic acid target sequences, as in many of the methods of the invention.

These novel probes are also useful for the detection of nucleic acid using the currently known and later-developed nucleic acid hybrid detection methods. Nucleic acid hybrid detection methods of the art are described in the background of the invention section hereinabove. Known nucleic acid hybrid detection methods include immobilization methods using labeled probe or hybrid (e.g. radioactive Southern blot), fluorescence quenching methods (e.g. Taqman®), and polymerization detection methods (primer extension methods).

The present invention provides a means for accurate measurement of the DNA of a primate, such as human, in samples that may contain DNA from various other sources—a problem commonly encountered in the forensic community. This problem has been addressed in the art using a variety of hybridization and amplification techniques, however, none of the techniques of the art have resulted in the development of a simple, rapid test that can accurately determine the amount of human DNA in a mixed DNA sample, either qualitatively or quantitatively. The present invention fills that need.

The present disclosure provides descriptions of methods and materials for the detection of a desired nucleic acid that contains multiple repeats of a predetermined nucleic acid target sequence. Such methods are useful for the rapid and accurate detection of a desired nucleic acid. In some embodiments, use of the present methods and materials can permit the detection of a desired nucleic acid without the need for enrichment of the nucleic acid target sequence within the nucleic acid of the sample, such as by PCR amplification. However, it may still be desirable to separate the nucleic acid from a sample, such as by nucleic acid precipitation. In some embodiments, the present methods and materials are desirable because they permit the detection and quantification of a specific organism's nucleic acid in a potentially mixed sample.

In the first embodiments of methods of the invention for determining the presence or absence of a desired nucleic acid that contains multiple repeats of a predetermined nucleic acid target sequence in a nucleic acid sample the steps comprise the following. A treated sample is provided that may contain the desired nucleic acid in which a plurality of the repeats of the predetermined repeating nucleic acid target sequence is hybridized with its corresponding nucleic acid probe. If the presence of a desired nucleic acid is detected, that presence is also quantifiable.

The invention contemplates a plurality of identical probes binding to a plurality of the multiple repeats of the predetermined nucleic acid target sequence on a single nucleic acid strand of the nucleic acid sample. For example, Alu sequences are repeated approximately 500,000 times in a haploid human genome (about 5×10⁹ bases). The repeated Alu sequences are themselves several hundred nucleic acid residues in length. The invention contemplates a nucleic acid probe to a portion of the Alu repeat. The invention also contemplates an embodiment wherein a plurality of different nucleic acid probes are able to hybridize to an Alu repeat at non-overlapping positions. The invention also contemplates an embodiment wherein two different nucleic acid probes are complementary to opposite strands in a region of the Alu repeat as their respective target sequences. The invention contemplates the use of both the preceding types of different probes (non-overlapping regions, different strands) in a single probesample hybridization treatment step.

A further consideration is that the ends of a repeated sequence, such as Alu, tend to be more highly conserved than the middle region of the repeated sequences, so in some embodiments it is preferable to design a probe to be complementary to a target region in the middle of the repeated sequence.

The treated sample is analyzed, quantitatively if desired, for the presence of hybridized nucleic acid containing the nucleic acid probe in order to deduce the presence or absence of said desired nucleic acid that contains multiple repeats of the predetermined nucleic acid target sequence.

In these embodiments, no further enrichment of the nucleic acid target sequence within the nucleic acid sample is typically necessary, because the target nucleic acid sample occurs with high enough frequency in the nucleic acid sample to be above the effective detection limit of a typical assay. In an embodiment where it is desired to avoid enrichment of the target nucleic acid within the nucleic acid, preferably the nucleic acid target sequence is repeated on average at least about once for every 100,000 bases. The present invention is also useful for nucleic acid target sequences repeated on average at least about once for every 10,000 bases in the desired nucleic acid, on average at least about once for every 3,000 bases in the desired nucleic acid, and on average at least about once for every 300 bases in the desired nucleic acid.

Each of the nucleic acid probes is about 10 to about 50 bases in length, preferably about 10 to about 30 bases in length and most preferably about 15 to about 25 bases in length. The nucleic acid probe comprises a portion that is at least about 10 nucleic acid residues in length and preferably at least about 15 nucleic acid residues in length that is complementary to all or a portion of the repeated nucleic acid target sequence. The nucleic acid probe is preferably complementary for its entire length to all or a portion of the repeated nucleic acid target sequence.

Many contemplated methods of analysis for hybridized nucleic acid are well known in the art. In addition, the methods described in the before-noted parental applications are also contemplated. Several known methods for nucleic acid hybrid detection are discussed in the Background of the Invention section hereinabove. The art discussed in that section is incorporated herein by reference for use in methods of the invention. Thus, contemplated analytical methods include the use of radiolabels, fluorescence spectroscopy, mass spectrometry, absorbance spectroscopy and luminescence spectroscopy. Illustrative contemplated analytical methods include observing a change in fluorescence quenching upon hybridization, labeled immobilization of hybrid, polymerization or depolymerization of hybridized probes.

In a second embodiment, a method of the invention for determining the presence or absence of a desired nucleic acid that contains multiple repeats of a predetermined nucleic acid target sequence in a nucleic acid sample the steps are as follows. A treated sample is provided that may contain the desired nucleic acid in which a plurality of the predetermined repeating nucleic acid target sequences are hybridized with a plurality of the same or different nucleic acid probes. The one or more predetermined nucleic acid target sequences are repeated on average preferably at least about once for every 100,000 bases. The present invention is also useful for nucleic acid target sequences repeated on average at least about once for every 10,000 bases in the desired nucleic acid, on average at least about once for every 3,000 bases in the desired nucleic acid, and on average at least about once for every 300 bases in the desired nucleic acid. When there is more than one predetermined nucleic acid target sequence used, the probes may differ from each other in length, sequence or both length and sequence. Also, each of the nucleic acid probes has a preferred length and complementarity to the nucleic acid target sequence as discussed before. Further, each of the nucleic acid probes is preferably specific to a particular source. The presence of hybridized nucleic acid containing said nucleic acid probe is analyzed for, optionally quantitatively, and thereby the presence or absence of the desired nucleic acid containing multiple repeats of the predetermined nucleic acid target sequence is ascertained.

Preferred organisms that can be detected using the materials and methods of the invention include homo sapiens (human), prokaryotes such as E. coli, and eukaryotes such as yeast.

Specificity is contemplated on a sliding scale according to the needs of the assay. If the goal is to distinguish between human and bacterial nucleic acid, then it is immaterial whether the probes used will distinguish between Escherichia coli and Escherichia blattae. The specificity required depends upon where on the phylogenetic tree the distinction is sufficient, be it a kingdom, phylum, class, order, family, genus or species. Bioinformatics comparisons and searches along with actual experiments are ideally used to select useful probes for task. The present disclosure provides several nucleic acid sequences that are useful for various applications that have already been selected and tested.

Preferred primate-specific nucleic acid probes include the sequences AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11) and their complements.

Preferred E. coli-specific nucleic acid probes include GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15) and their complements.

Preferred yeast-specific nucleic acid probes include AAGATGACGCAAATGATG (SEQ ID NO:16); GAAGATGACGCAAATGAT (SEQ ID NO:17) and their complements.

The invention contemplates a nucleic acid probe that is species-specific, preferably about 10 to about 50 bases in length, more preferably about 10 to about 30 bases in length and most preferably about 15 to about 25 bases in length and comprises a nucleic acid sequence listed above.

In a third embodiment, a method of the invention for determining the presence or absence of a desired nucleic acid that contains multiple repeats of a predetermined nucleic acid target sequence in a nucleic acid sample is comprised of the following steps. A treated sample is provided that may contain said predetermined nucleic acid target sequence hybridized with a nucleic acid probe that includes an identifier nucleotide in the 3′-terminal region. The treated sample is admixed with a depolymerizing amount of an enzyme whose activity is to release one or more nucleotides from the 3′-terminus of a hybridized nucleic acid probe to form a treated reaction mixture. The treated reaction mixture is maintained for a time period sufficient to permit the enzyme to depolymerize hybridized nucleic acid and release identifier nucleotides therefrom. The presence of released identifier nucleotides is analyzed for to obtain an analytical output. The analytical output, quantitatively if desired, indicates the presence or absence of the predetermined nucleic acid target sequence and thus the desired nucleic acid that contains multiple repeats of the predetermined nucleic acid target sequence. In a preferred embodiment, the depolymerizing enzyme is a template-dependent polymerase including, but not limited to, AMV reverse transcriptase, MMLV reverse transcriptase, DNA polymerase alpha or beta, Taq polymerase, Tth polymerase, Tne polymerase, Tne triple mutant polymerase, Tvu polymerase, Ath polymerase, E. coli DNA polymerase I, T4 DNA polymerase, Klenow fragment, Klenow exo minus, or poly(A) polymerase.

In a fourth set of embodiments, method steps are included for formation of a treated sample that may contain the predetermined nucleic acid target sequence hybridized with a nucleic acid probe by admixing a sample to be assayed with one or more nucleic acid probes to form a hybridization composition. Here, the 3′-terminal region of the nucleic acid probes (i) hybridize with total complementarity to said predetermined nucleic acid target sequence when that sequence is present in the sample and preferably (ii) include an identifier nucleotide as discussed hereinafter.

In a fifth set of embodiments, a method of the invention for determining the presence or absence of a desired nucleic acid that contains multiple repeats of a predetermined nucleic acid target sequence in a nucleic acid sample is conducted in a convenient, optionally quantitative, one-pot method comprising the following steps. A treated sample that may contain the predetermined nucleic acid target sequence hybridized to a nucleic acid probe is admixed with (i) a depolymerizing amount of an enzyme whose activity in the presence of pyrophosphate is to release identifier nucleotide as a nucleoside triphosphate from the hybridized nucleic acid probe, (ii) adenosine 5′ diphosphate, (iii) pyrophosphate and (iv) NDPK to form a treated reaction mixture. The 3′-terminal region of the nucleic acid probe is completely complementary to said predetermined nucleic acid target sequence and includes an identifier nucleotide. The treated reaction mixture is maintained at a temperature of about 25 to about 80 degrees C. for a time period sufficient to permit the enzyme to depolymerize hybridized nucleic acid probe, release an identifier nucleotide in the 3′-terminal region as a nucleoside triphosphate and to convert the nucleoside triphosphate and the adenosine 5′ diphosphate to adenosine 5′ triphosphate. The presence of adenosine 5′ triphosphate is analyzed for in order to obtain an analytical output. The analytical output indicates the presence or absence of at least one of the nucleic acid target sequences. Preferably, the analytical output from ATP analysis is obtained by luminescence spectroscopy, most preferably using a luciferase/luciferin detection system. The analytical output is optionally used to quantify the presence of the desired nucleic acid.

A subset of the fifth set of embodiments includes forming the treated sample. Thus, a sample to be assayed is admixed with one or more nucleic acid probes to form a hybridization composition, wherein the 3′-terminal region of said nucleic acid probe (i) hybridizes with total complementarity to a nucleic acid target sequence when that sequence is present in the sample and (ii) includes an identifier nucleotide. The hybridization composition is maintained for a time period sufficient to form a treated sample that may contain the predetermined nucleic acid target sequence hybridized with a nucleic acid probe.

In a subset of the methods of the fifth embodiment of the invention, the depolymerizing enzyme maintains activity at 60-90° C. Preferably, the depolymerizing enzyme is Tne triple mutant DNA polymerase, Bst DNA polymerase, Ath DNA polymerase, Taq DNA polymerase or Tvu DNA polymerase.

In a subset of the methods of the fifth embodiment of the invention, preferably the NDPK is that encoded by Pyrococcus furiosis.

The invention also contemplates a first kit embodiment for determining the presence or absence of a desired nucleic acid that contains multiple repeats of a predetermined nucleic acid target sequence in a nucleic acid sample in which the nucleic acid target sequence is repeated on average preferably at least about once for every 100,000 bases in the desired nucleic acid. The present invention is also useful for nucleic acid target sequences repeated on average at least about once for every 10,000 bases in the desired nucleic acid, on average at least about once for every 3,000 bases in the desired nucleic acid, and on average at least about once for every 300 bases in the desired nucleic acid. The first kit embodiment comprises a package containing at least one nucleic acid probe that is (i) about 10 to about 50 bases in length, and (ii) complementary to all or a portion of said repeated nucleic acid target sequence, and instructions for use. In a subset of the first kit embodiments, the kit contains a plurality of different nucleic acid probes. In the first kit embodiments, the nucleic acid probe is preferably about 10 to about 50 bases in length, more preferably about 10 to about 30 bases in length and most preferably about 15 to about 25 bases in length.

In a subset of the first kit embodiments, the nucleic acid probe includes a nucleic acid sequence AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11); GAATCCCCAGGAGCTTACATA (SEQ ID NO:12; CCCAGGAGCTTACATA (SEQ ID NO:13); GTGACCGGGGTGAGGGCGTG (SEQ ID NO:14; AGTGACTGGGG (SEQ ID NO:15; AAGATGACGCAAATGATG (SEQ ID NO:16); GAAGATGACGCAAATGAT (SEQ ID NO:17) or a sequence complementary to those listed sequences.

In a subset of the first kit embodiments, the nucleic acid probe includes a nucleic acid sequence of AGACCCCATCTCTAA (SEQ ID NO:l); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11) or a sequence complementary to those listed sequences.

In a subset of the first kit embodiments, the nucleic acid probe includes a nucleic acid sequence of GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15) or a sequence complementary to those listed sequences.

In a subset of the first kit embodiments, the nucleic acid probe includes a nucleic acid sequence of AAGATGACGCAAATGATG (SEQ ID NO:16); GAAGATGACGCAAATGAT (SEQ ID NO:17) or a sequence complementary to those listed sequences.

In a subset of the first kit embodiments, the kit also includes an enzyme whose activity is to release one or more nucleotides from the 3′ terminus of a hybridized nucleic acid probe.

In a subset of the first kit embodiments, the kit also includes adenosine 5′ diphosphate, pyrophosphate, and a nucleoside diphosphate kinase (NDPK).

The present invention has many benefits and advantages, several of which are listed below.

One benefit of some embodiments of the invention is that they permit nucleic acid detection based on the use of probes for repeated sequences that provide very high levels of sensitivity without the need for radiochemicals or electrophoresis.

An advantage of some embodiments of the invention is that the presence or absence of one or more repeated nucleic acid target sequences can be detected reliably, reproducibly, and with great sensitivity.

A further benefit of some embodiments of the invention is that quantitative information can be obtained about the amount of a repeated nucleic acid target sequence in a sample.

A further advantage of some embodiments of the invention is that the use of repeated sequences as nucleic acid target sequences permits sensitive detection without a need for enrichment of a target-containing segment of nucleic acid from an entire nucleic acid sample, such as genomic DNA.

Yet another benefit of some embodiments of the invention is that the nucleic acid probes of the invention permit source-specific nucleic acid detection.

Yet another advantage of some embodiments of the invention is that the presence or absence of a nucleic acid target sequence can be determined with a small number of reagents and manipulations.

Another benefit of some embodiments of the invention is that the processes lend themselves to automation.

Still another benefit of several embodiments of the invention is their flexibility of use in many different types of applications and assays including, but not limited to, detection of specific DNA from the nucleic acid of a biological sample, species identification, sample contamination, and analysis of forensic samples.

Still further benefits and advantages of the invention will become apparent from the specification and claims that follow.

Definitions

To facilitate understanding of the invention, a number of terms are defined below.

A “nucleic acid,” as used herein, is a covalently linked sequence of nucleotides in which the 3′ position of the pentose of one nucleotide is joined by a phosphodiester group to the 5′ position of the pentose of the next, and in which the nucleotide residues (bases) are linked in specific sequence; i.e., a linear order of nucleotides. A “polynucleotide,” as used herein, is a nucleic acid containing a sequence that is greater than about 100 nucleotides in length. An “oligonucleotide,” as used herein, is a short polynucleotide or a portion of a polynucleotide. An oligonucleotide typically contains a sequence of about two to about one hundred bases. The word “oligo” is sometimes used in place of the word “oligonucleotide”.

A “nucleic acid of interest” or “desired nucleic acid” as used herein, is any particular nucleic acid one desires to study in a sample.

The term “isolated” when used in relation to a nucleic acid or protein, refers to a nucleic acid sequence or protein that is identified and separated from at least one contaminant (nucleic acid or protein, respectively) with which it is ordinarily associated in its natural source. Isolated nucleic acid or protein is present in a form or setting that is different from that in which it is found in nature. In contrast, non-isolated nucleic acids or proteins are found in the state they exist in nature.

As used herein, the term “purified” or “to purify” means the result of any process which removes some contaminants from the component of interest, such as a protein or nucleic acid. The percent of a purified component is thereby increased in the sample.

Nucleic acids are known to contain different types of mutations. A “lesion”, as used herein, refers to site within a nucleic acid where one or more bases are mutated by deletion or insertion, so that the nucleic acid sequence differs from the wild-type sequence. The term “wild-type,” as used herein, refers to a gene or gene product that has the characteristics of that gene or gene product that is most frequently observed in a population and is thus arbitrarily designated the “normal” or “wild-type” form of the gene.

DNA molecules are said to have a “5′-terminus” (5′ end) and a “3′-terminus” (3′ end) because nucleic acid phosphodiester linkages occur to the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′- or 5′-terminus. As used herein, a nucleic acid sequence, even if internal to a larger oligonucleotide or polynucleotide, also can be said to have 5′- and 3′- ends. For example, a gene sequence located within a larger chromosome sequence can still be said to have a 5′- and 3′-end.

As used herein, the 3′-terminal region of the nucleic acid probe refers to the region of the probe including nucleotides within about 10 residues from the 3′-terminal position.

In either a linear or circular DNA molecule, discrete elements are referred to as being “upstream” or “5′” relative to an element if they are bonded or would be bonded to the 5′-end of that element. Similarly, discrete elements are “downstream” or “3′” relative to an element if they are or would be bonded to the 3′-end of that element. Transcription proceeds in a 5′ to 3′ manner along the DNA strand. This means that RNA is made by the sequential addition of ribonucleotide-5′-triphosphates to the 3′-terminus of the growing chain (with the elimination of pyrophosphate).

As used herein, the term “target nucleic acid sequence” or “nucleic acid target sequence” refers to a particular nucleic acid sequence of interest. Thus, the “target” can exist in the presence of other nucleic acid molecules or within a larger nucleic acid molecule.

As used herein, the term “nucleic acid probe” refers to an oligonucleotide or polynucleotide that is capable of hybridizing to another nucleic acid of interest. A nucleic acid probe may occur naturally as in a purified restriction digest or be produced synthetically, recombinantly or by PCR amplification. As used herein, the term “nucleic acid probe” refers to the oligonucleotide or polynucleotide used in a method of the present invention. That same oligonucleotide could also be used, for example, in a PCR method as a primer for polymerization, but as used herein, that oligonucleotide would then be referred to as a “primer”. Herein, oligonucleotides or polynucleotides may contain a phosphorothioate bond.

As used herein, the terms “complementary” or “complementarity” are used in reference to nucleic acids (i.e., a sequence of nucleotides) related by the well-known base-pairing rules that A pairs with T and C pairs with G. For example, the sequence 5′-A-G-T-3′, is complementary to the sequence 3′-T-C-A-5′. Complementarity can be “partial,” in which only some of the nucleic acid bases are matched according to the base pairing rules. On the other hand, there may be “complete” or “total” complementarity between the nucleic acid strands when all of the bases are matched according to base pairing rules. The degree of complementarity between nucleic acid strands has significant effects on the efficiency and strength of hybridization between nucleic acid strands as known well in the art. This is of particular importance in detection methods that depend upon binding between nucleic acids, such as those of the invention. The term “substantially complementary” refers to any probe that can hybridize to either or both strands of the nucleic acid target sequence under conditions of low stringency as described below or, preferably, in polymerase reaction buffer (Promega, M195A) heated to 95° C. and then cooled to room temperature. As used herein, when the nucleic acid probe is referred to as partially or totally complementary to the nucleic acid target sequence, that refers to the 3′-terminal region of the probe (i.e. within about 10 nucleotides of the 3′-terminal nucleotide position).

As used herein, the term “hybridization” is used in reference to the pairing of complementary nucleic acid strands. Hybridization and the strength of hybridization (i.e., the strength of the association between nucleic acid strands) is impacted by many factors well known in the art including the degree of complementarity between the nucleic acids, stringency of the conditions involved affected by such conditions as the concentration of salts, the Tₘ (melting temperature) of the formed hybrid, the presence of other components (e.g., the presence or absence of polyethylene glycol), the molarity of the hybridizing strands and the G:C content of the nucleic acid strands.

As used herein, the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids which are not completely complementary to one another be hybridized or annealed together. The art knows well that numerous equivalent conditions can be employed to comprise low stringency conditions.

The term “homology,” as used herein, refers to a degree of complementarity. There can be partial homology or complete homology (i.e., identity). A partially complementary sequence that at least partially inhibits a completely complementary sequence from hybridizing to a nucleic acid target sequence is referred to using the functional term “substantially homologous.” A substantially homologous probe hybridizes to a nucleic acid strand under conditions of low stringency.

The term “identifier nucleotide,” as used herein, refers to a nucleotide whose presence is to be detected in a process of the invention to identify that a depolymerization reaction has occurred. The particular application of a method of the invention affects which residues are considered an identifier nucleotide. For a method using ATP detection (e.g. luciferase/luciferin or NADH) wherein, during analysis, all nucleotides released in the depolymerization are “converted” to ATP with an enzyme such as NDPK, all nucleotides released are identifier nucleotides. Similarly, for a method using absorbance detection that does not distinguish between nucleotides, all released nucleotides are identifier nucleotides. For a mass spectrometric detection wherein all the released nucleotides are analyzed, all released nucleotides can be identifier nucleotides; alternatively a particular nucleotide (e.g. a nucleotide analog having a distinctive mass) can be detected. For fluorescence detection, a fluorescently-labeled nucleotide is an identifier nucleotide. The nucleotide may be labeled prior to or after release from the nucleic acid. For radiographic detection, a radioactively-labeled nucleotide is an identifier nucleotide. In some cases, the release of identifier nucleotide is deduced by analyzing the remainder of the probe after a depolymerization step of the invention. Such analysis is generally by a determination of the size or mass of the remaining probe and can be by any of the described analytical methods (e.g. a fluorescent tag on the 5′-terminus of the probe to monitor its molecular weight following capillary electrophoresis).

The term “sample,” as used herein, is used in its broadest sense. A sample suspected of containing a nucleic acid can comprise a cell, chromosomes isolated from a cell (e.g., a spread of metaphase chromosomes), genomic DNA, RNA, cDNA and the like.

The term “detection,” as used herein, refers to quantitatively or qualitatively identifying a nucleotide or nucleic acid within a sample.

The term “depolymerization,” as used herein, refers to the removal of a nucleotide from the 3′ end of a nucleic acid.

### DETAILED DESCRIPTION OF THE INVENTION

The present disclosure describes several nucleic acid sequences that are useful for the detection of nucleic acid in a sample in an embodiment of the present invention. These sequences have advantage over sequences disclosed in the art because they are probes to target sequences that are present at high enough copy number in a raw sample that it is possible to detect the presence of a nucleic acid without the need to undergo a nucleic acid target sequence enrichment step prior to a nucleic acid hybrid detection step.

Nucleic acid target sequence enrichment steps include cutting nucleic acid in a raw sample and separating nucleic acid containing the target sequence from nucleic acid that does not contain the target sequence. For example, this might entail amplifying a portion of the nucleic acid containing the target sequence. Another kind of target enrichment step would be digestion with a restriction endonuclease and isolation of the fragment containing the target sequence.

The repetitive sequences of the present invention are particularly useful for source-specific nucleic acid detection. As used herein, the term “source-specific” loosely describes probes useful for distinguishing between organisms that may be present in a sample. The nucleic acid of the source has a repeating nucleic acid sequence that will hybridize with a contemplated source-specific probe. In some embodiments, a source-specific probe will hybridize effectively to all members of a single species (e.g. homo sapiens). In other embodiments, a source-specific probe will hybridize effectively to all members of a genus. In other embodiments, a source-specific probe will hybridize effectively to all members of an order. And, in still other embodiments, a source-specific probe will hybridize effectively to all members of a family.

In some embodiments, a source-specific probe will hybridize effectively with one group of organisms and not another, for example a probe that will hybridize with primates but not with other mammals, or a probe that will hybridize with all mammals but not other life forms.

A probe is still considered “source-specific” even if there is some cross-hybridization with other organisms, such as a primate-specific probe that may cross-hybridize with cows. Such a probe is still useful for distinguishing between, for example, human and bacterial sources of nucleic acid. Some repetitive elements and their natural occurrences are discussed hereinbelow.

The selected repetitive sequences are present in high copy number in the organism or source of interest. Preferably, the analytical output is at least two-fold, more preferably five-fold, and even more preferably ten-fold, over the sample controls including nucleic acid from organisms that are not being detected (i.e. low analytical output).

Situations where source-specific nucleic acid detection is useful include determining the source of an unknown nucleic acid sample or detection of the presence or identity of a contaminant. A forensic laboratory might use source-specific nucleic acid detection to ascertain whether blood came from a human or an animal. A food science laboratory might use source-specific nucleic acid detection to ascertain whether a beef or chicken sample is contaminated with E. coli.

Although one of the advantages of the methods of the present invention using highly a repetitive nucleic acid target sequence to detect the presence of a nucleic acid is that sensitivity enhancement via enrichment (e.g. amplification) of the nucleic acid target sequence is not necessary, there may be situations where enrichment is desirable, depending on the desired sensitivity of the assay.

The method of this embodiment of the present invention uses highly repetitive sequences as targets for sensitive nucleic acid detection. Novel probes are provided that form nucleic acid hybrids with their respective nucleic acid target sequences, as in many of the methods of the invention.

These novel probes are also useful for the detection of nucleic acid using the currently known and later-developed nucleic acid hybrid detection methods. Nucleic acid hybrid detection methods of the art are described in the background of the invention section hereinabove and in the patent applications related to the present applications regarding depolymerization detection methods (e.g. READIT™ technology, Promega Corp.). Known nucleic acid hybrid detection methods include immobilization methods using labeled probe or hybrid (e.g. radioactive Southern blot), fluorescence quenching methods (e.g. Taqman®), and polymerization detection methods (primer extension methods). The READIT™ detection technology can also be utilized in immobilization methods, fluorescence quenching methods and polymerization methods.

Many depolymerization methods and materials for detecting nucleic acid hybrids are disclosed in the following Promega Corp. patent applications and publications, the disclosures of all of which are incorporated in full herein by reference: WO 99/46409 “Nucleic Acid Detection,” published Sep. 16, 1999 corresponding to U.S. patent application Ser. No. 09/252,436, filed on Feb. 18, 1999, now U.S. Pat. No. 6,159,693 issued Dec. 12, 2000; WO 00/49180 “Detection of Nucleic Acid Hybrids,” published Aug. 24, 2000, corresponding to U.S. patent application Ser. No. 09/383,316 filed Aug. 25, 1999; WO 00/49179 “Methods for determining the presence of nucleic acid target sequences and applications thereof,” published Aug. 24, 2000 corresponding to U.S. patent application Ser. No. 09/406,147 filed Sep. 27, 1999; WO 00/49181 “Multiplex Method for Nucleic Acid Determination,” published Aug. 24, 2000 corresponding to allowed U.S. patent application Ser. No. 09/406,064 filed Sep. 27, 1999; WO 00/49182 “Analytical Methods and Materials for Nucleic Acid Detection,” published Aug. 24, 2000, corresponding to allowed U.S. patent application Ser. No. 09/425,460 filed Oct. 22, 1999; allowed U.S. patent application Ser. No. 09/358,972, “Detection of Nucleic Acid Hybrids,” published Feb. 18, 2000 at http://www.promega.com/pt/pend/09358972.pdf; allowed U.S. patent application Ser. No. 09/406,065 “Improved Nucleic Acid Detection,” published Feb. 18, 2000 at http://www.Promega.com/pt/pend/09406065.pdf; U.S. patent application Ser. No. 09/430,615, “Method for Amplified Nucleic Acid Detection,” published Feb. 18, 2000 at http://www.Promega.com/pt/pend/09430615.pdf.

The present disclosure provides descriptions of methods and materials for the detection of a desired nucleic acid that contains multiple repeats of a predetermined nucleic acid target sequence. Such methods are useful for the rapid and accurate detection of a desired nucleic acid. In some embodiments, use of the present methods and materials can permit the detection of a desired nucleic acid without the need for enrichment of the nucleic acid target sequence within the sample. In some embodiments, the present methods and materials are desirable because they permit the organism-specific detection, and optionally quantification, of a desired nucleic acid.

The present invention contemplates several repeated sequences that are useful for species-specific nucleic acid detection. The contemplated sequences are useful for determination of the source of the nucleic acid sample using specific probes and nucleic acid hybrid detection methods of the art and of the invention.

In the first embodiments of methods of the invention for determining the presence or absence of a desired nucleic acid that contains multiple repeats of a predetermined nucleic acid target sequence in a nucleic acid sample the steps comprise the following. A treated sample is provided that may contain the desired nucleic acid in which a plurality of the predetermined repeating nucleic acid target sequences is hybridized with nucleic acid probes.

The treated sample is analyzed for the presence of hybridized nucleic acid containing the nucleic acid probe in order to deduce the presence or absence of said desired nucleic acid that contains multiple repeats of the predetermined nucleic acid target sequence.

In the human genome, interspersed repeats have been classified in at least four distinct families, Alu, LINE 1, MIR and MaLR, that make up approximately 10-15 percent of the human genome. The families differ in their retropositional activity. Useful reviews on Alu repeats, incorporated herein by reference, are Carl W. Schmid, “Alu: Structure, Origin, Evolution, Significance, and Function of One-Tenth of Human DNA,” Prog. in Nucl. Acid Res. and Mol. Biol., 53:283-319 (1996); Hamdi Hamdi, et al., “Origin and Phylogenetic Distribution of Alu DNA Repeats: Irreversible Events in the Evolution of Primates,” J. Mol. Biol. (1999); P. L. Deininger and M. A. Batzer, “Alu Repeats and Human Disease,” Mol. Genet. Metab., 67(3):183-193 (1999) (Alu element copy number is in excess of 500,000 copies per haploid human genome); and Mark. A. Batzer, et al., “Standardized Nomenclature for Alu Repeats,” J. Mol. Evol., 42:3-6 (1996). The genomes of eukaryotes also contain short interspersed elements, SINEs.

Targets contemplated for use in methods of the invention include nucleic acid target sequences that are known to be repeated multiple times in a desired nucleic, such as those known as Alu and LINES. There is a great deal of literature regarding repetitive sequences. A useful review of Alu is the Schmid article, cited above. A useful review of LINEs is P. Deininger, et al., Trend Genet., 8:307 (1992).

Interspersed repetitive sequences are major components of eukaryotic genomes. Repetitive elements can make up nearly a half of a genome, e.g., 45% of the silkworm (Bombyx mori). Some repetitive sequences are lineage-specific, others are common in a wide spectrum of organisms, which suggests that the former are more recent than the latter in terms of their evolutionary origin. Among different categories of interspersed repetitive elements, the most abundant in eukaryotic genomes are long and short interspersed elements (LINE or long interspersed repetitive elements, and SINEs or short interspersed repetitive elements, respectively). The SINE families are the most highly repeated elements in eukaryotic genomes. The well-characterized SINE families have several features in common. By definition, all SINEs are small, up to 1000 nucleotides in length. They are present in tens to hundred of thousands of copies per genome, and a single species may have more than one SINE family. SINEs evolved multiple times in eukaryotes from the genes coding for small, untranslated RNAs such as 7SL RNA or tRNA.

Preferably, the target sequence is repeated a sufficient number of times in the desired nucleic acid that further target enrichment methods are unnecessary. No further enrichment of the nucleic acid target sequence within the nucleic acid sample is typically necessary with the presently disclosed species-specific nucleic acid probe sequences, because the nucleic acid target sequence occurs with high enough frequency in the nucleic acid sample to be above the effective detection limit of a typical assay. Preferably, the nucleic acid target sequence is repeated on average at least about once for every 100,000 bases in the desired nucleic acid. The present invention is also useful for nucleic acid target sequences repeated on average at least about once for every 10,000 bases in the desired nucleic acid, on average at least about once for every 3,000 bases in the desired nucleic acid, and on average at least about once for every 300 bases in the desired nucleic acid.

The selection of other nucleic acid target sequences that are repeated multiple times within a desired nucleic acid are typically selected by searching nucleic acid databases. For example, a genome is searched for the number of repeats of various proposed repeated target sequences, typically starting with short strings of about 8 nucleic acid residues. Additional bases are then added to sequences that are repeated a sufficient number of times to determine the effect on the number of occurrences within a genome. The process is iterated to arrive at candidate nucleic acid target sequences.

Each of the nucleic acid probes is preferably about 10 to about 50 bases in length, more preferably about 10 to about 30 bases in length and most preferably about 15 to about 25 bases in length. The nucleic acid probe comprises a portion that is at least about 10 nucleic acid residues in length and preferably at least about 15 nucleic acid residues in length that is complementary to all or a portion of the repeated nucleic acid target sequence. The nucleic acid probe is preferably is complementary for its entire length to all or a portion of the repeated nucleic acid target sequence.

Many contemplated methods of analysis for hybridized nucleic acid are well known in the art. In addition, the methods described in the before-noted Promegaparental applications are also contemplated. Such methods include the reagents and methods for detecting nucleic acid hybrids by depolymerizing a probe/hybrid complex (by pyrophosphorolysis or exonuclease digestion) and then determining whether depolymerization occurred by any of a wide variety of methods. Methods to determine whether depolymerization occurred typically involve either analysis of the released nucleotides or the remaining probe or target strand from which nucleotides were released. For example, fluorescent or radioactive or mass-differentiating markers on either the released nucleotide(s) or remaining probe can provide this information. Fluorescence quenching changes from a sequence having a fluorescent donor/quencher pair will provide evidence that the donor and quencher have been separated by depolymerization. Depolymerization of an intentional mismatch at the 3′ terminus of a probe can also be detected by observing subsequent polymerization initiation from that probe under polymerizing conditions. Phosphate transfer reactions can be used to detect released nucleotides by transfer of phosphate groups to substrates (such as NADPH or ATP) for detecting enzymes, such as luciferase (in the presence of luciferin). Other methods of detecting hybridization are included in the incorporated Promega patent applications, including various methods to amplify either the target for hybridization based on the presence of a desired nucleic acid target, or methods and materials to amplify the substrates such as ATP.

Several known methods for nucleic acid hybrid detection are discussed in the Background of the Invention section hereinabove. The art discussed in that section is incorporated herein by reference for use in methods of the invention. Thus, contemplated analytical methods include the use of radiolabels, fluorescence spectroscopy, mass spectrometry, absorbance spectroscopy and luminescence spectroscopy. Illustrative contemplated analytical methods include observing a change in fluorescence quenching upon hybridization, labeled immobilization of hybrid, polymerization or depolymerization of hybridized probes. Another contemplated analytical method is PCR amplification subsequent to depolymerization of the probe, using the modified probe as a PCR primer.

For methods of the first embodiment, an ordinary practitioner in the art recognizes that substantially any method for the detection of nucleic acid hybrids can be used in contemplated method. Such a practitioner typically considers such factors as sample size, sensitivity, time limitations, as they pertain to a particular situation in selecting an analytical method for nucleic acid hybrid detection.

Absorption spectroscopic analysis of light produced from a luciferin/luciferase reaction with ATP formed from released nucleotides (preferably catalyzed by a nucleotide diphosphate kinase) is preferred where rapid, sensitive nucleic acid detection is desired, such as READIT™. Alternative preferred embodiments are flourescence quenching techniques, such as Taqman®.

Variations of nucleic acid hybrid detection are contemplated. For example, fluorescent tags can be introduced before or after hybrid formation. Flourescent detection can be direct or indirect. Mass spectrometric methods are flexible and can be used to distinguish extended or depolymerized primers in addition to incorporation or removal of bases of differing mass units. Recent improvements in mass spectrometric techniques have been reducing sample size requirements. Several of the techniques are amenable to the conduct of multiple experiments at once, as in a microarray and/or robotic format.

The methods disclosed herein are useful with a variety of samples, but typically, the nucleic acid sample is obtained from a biological sample.

When the analytical output is obtained by fluorescence spectroscopy, the fluorescent tag is either already on a strand of the nucleic acid hybrid or it is labeled later, and the fluorescent label is direct or indirect. For example, in a depolymerization embodiment, the fluorescent tag in one embodiment is on a nucleotide released by depolymerization, in another embodiment, the fluorescent tagremains on the hybrid after depolymerization, and in yet another embodiment one partner of a fluorescence donor/quencher pair is on the nucleotide that is released. In a primer extension embodiment, the label is typically incorporated in the extension reaction. In one type of depolymerization embodiment the released identifier nucleotide includes a fluorescent label that is optionally labeled after release from the hybrid.

The invention contemplates desired nucleic acid sequences wherein a repeated sequence in the desired nucleic acid includes a plurality of repeated sequences that differ in length. For example, there may be some variation in the length of the various repeats of a repeated Alu sequence throughout a particular individual's genome, but a person of skill in the art recognizes each of those versions as slight differences in a repeated Alu sequence.

The invention further contemplates a plurality of predetermined repeated sequences differ in sequence. For example, there may be some variation in the sequence of the various repeats of a repeated Alu sequence throughout a particular individual's genome, but a person of ordinary skill in the art recognizes each of those versions as slight differences in a repeated Alu sequence. The invention further contemplates a repeated sequence of the desired nucleic acid that includes a plurality of predetermined repeated sequences that differ in both length and sequence.

In some embodiments of the invention, the nucleic acid target sequences are selected such that they are complementary to a portion of the repeating sequence that is unaffected by variation in length or sequence. In some embodiments of the invention, the nucleic acid target sequences are selected such that they are substantially complementary to a portion of the repeating sequence, and effective hybrids are still formed despite variation in sequence occurring within the portion of the repeating sequence that is complementary to a nucleic acid probe sequence.

The invention further contemplates nucleic acid probes that are source-specific, as discussed herein.

In a second embodiment, a method of the invention for determining the presence or absence of a desired nucleic acid that contains multiple repeats of a predetermined nucleic acid target sequence in a nucleic acid sample the steps are as follows. A treated sample is provided that may contain the desired nucleic acid in which a plurality of the predetermined repeating nucleic acid target sequences are hybridized with a plurality of the same or different nucleic acid probes.

The one or more predetermined nucleic acid target sequences is repeated on average preferably at least about once for every 100,000 bases in the desired nucleic acid. The present invention is also useful for nucleic acid target sequences repeated on average at least about once for every 10,000 bases in the desired nucleic acid, on average at least about once for every 3,000 bases in the desired nucleic acid, and on average at least about once for every 300 bases in the desired nucleic acid.

When a plurality of nucleic acid probes is used, the nucleic acid probes may differ from each other in length, sequence or both length and sequence. Also, each of the nucleic acid probes has a preferred length and complementarity to the nucleic acid target sequence as discussed before. Further, each of the nucleic acid probes is preferably source-specific. The presence of hybridized nucleic acid containing said nucleic acid probe is analyzed for, and thereby the presence or absence of the desired nucleic acid containing multiple repeats of the predetermined nucleic acid target sequence is ascertained.

Preferred species that can be detected using the materials and methods of the invention include mammals such as humans (homo sapiens), prokaryotes such as E. coli, and eukaryotes such as yeast (S. cerevisiae).

Preferred primate-specific nucleic acid probes include AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11) and their complements.

Preferred E. coli-specific nucleic acid probes include GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15) and their complements.

Preferred yeast-specific nucleic acid probes include AAGATGACGCAAATGATG (SEQ ID NO:16); GAAGATGACGCAAATGAT (SEQ ID NO:17) and their complements.

The invention contemplates departures from the sequences of the probes or their complementary targets disclosed herein that are still useful in a process of the invention, as long as they still form nucleic acid hybrids under the conditions used for the formation of a “treated sample”. Specifically, “lesions” such as single base insertions or deletions or base changes are contemplated, particularly when such lesions are not within about four bases and preferably not within about ten bases of the actual 3′-terminal nucleotide of the nucleic acid probe.

The invention contemplates a nucleic acid probe that is species-specific, has a preferable length of about 10 to about 50 bases, more preferably about 10 to about 30 bases in length and most preferably about 15 to about 25 bases in length and includes a nucleic acid sequence listed above. Preferably, the other nucleic acid bases in a probe including one of the above-listed sequences have a sequence of one, and more preferably many, of the repeats in the nucleic acid of interest.

In a third embodiment, a method of the invention for determining the presence or absence of a desired nucleic acid that contains multiple repeats of a predetermined nucleic acid target sequence in a nucleic acid sample is comprised of the following steps. A treated sample is provided that may contain said predetermined nucleic acid target sequence hybridized with a nucleic acid probe that includes an identifier nucleotide in the 3′-terminal region. The treated sample is admixed with a depolymerizing amount of an enzyme whose activity is to release one or more nucleotides from the 3′-terminus of a hybridized nucleic acid probe to form a treated reaction mixture. The treated reaction mixture is maintained for a time period sufficient to permit the enzyme to depolymerize hybridized nucleic acid and release identifier nucleotides therefrom. The presence of released identifier nucleotides is analyzed to obtain an analytical output. The analytical output indicates the presence or absence of the predetermined nucleic acid target sequence and thus the desired nucleic acid that contains multiple repeats of the predetermined nucleic acid target sequence. Preferably, in the third embodiment, the analytical output is obtained by the various methods described for depolymerization hereinbelow.

In a fourth set of embodiments, method steps are included for formation of a treated sample that may contain the predetermined nucleic acid target sequence hybridized with a nucleic acid probe by admixing a sample to be assayed with one or more nucleic acid probes to form a hybridization composition. Here, the 3′-terminal region of the nucleic acid probes (i) hybridize with total complementarity to said predetermined nucleic acid target sequence when that sequence is present in the sample and preferably (ii) include an identifier nucleotide as discussed before and hereinafter.

In a fifth set of embodiments, a method of the invention for determining the presence or absence of a desired nucleic acid that contains multiple repeats of a predetermined nucleic acid target sequence in a nucleic acid sample is conducted in a convenient one-pot method comprising the following steps. A treated sample that may contain the predetermined nucleic acid target sequence hybridized to a nucleic acid probe is admixed with (i) a depolymerizing amount of an enzyme whose activity in the presence of pyrophosphate is to release identifier nucleotide as a nucleoside triphosphate from the hybridized nucleic acid probe, (ii) adenosine 5′ diphosphate, (iii) pyrophosphate and (iv) NDPK to form a treated reaction mixture. The 3′-terminal region of the nucleic acid probe is completely complementary to said predetermined nucleic acid target sequence and includes an identifier nucleotide. The treated reaction mixture is maintained at a temperature of about 25 to about 80 degrees C. for a time period sufficient to permit the enzyme to depolymerize hybridized nucleic acid probe, release an identifier nucleotide in the 3′-terminal region as a nucleoside triphosphate and to convert the nucleoside triphosphate and the adenosine 5′ diphosphate to adenosine 5′ triphosphate. The presence of adenosine 5′ triphosphate is analyzed for in order to obtain an analytical output. The analytical output indicates the presence or absence of at least one of the nucleic acid target sequences. Preferably, the analytical output from ATP analysis is obtained by luminescence spectroscopy, most preferably using a luciferase/luciferin detection system.

A subset of the fifth set of embodiments includes forming the treated sample. Thus, a sample to be assayed is admixed with one or more nucleic acid probes to form a hybridization composition, wherein the 3′-terminal region of said nucleic acid probe (i) hybridizes with total complementarity to a nucleic acid target sequence when that sequence is present in the sample and (ii) includes an identifier nucleotide. The hybridization composition is maintained for a time period sufficient to form a treated sample that may contain the predetermined nucleic acid target sequence hybridized with a nucleic acid probe.

In a subset of the methods of the fifth embodiment of the invention, the depolymerizing enzyme maintains activity at 60-90° C. Preferably, the depolymerizing enzyme is Tne triple mutant DNA polymerase, Bst DNA polymerase, Ath DNA polymerase, Taq DNA polymerase or Tvu DNA polymerase. The Tne triple mutant DNA polymerase is described in detail in WO 96/41014, whose disclosures are incorporated by reference, and its 610 residue amino acid sequence is provided as SEQ ID NO:35 of that document. That enzyme is referred to in WO 96/41014 as Tne M284 (D323A,D389A).

In a subset of the methods of the fifth embodiment of the invention, preferably the NDPK is that encoded by Pyrococcus furiosis. Although yeast, bovine or another NDPK can be used in these reactions, it is preferred to utilize a thermostable NDPK such as the Pfu NDPK described in Example 24 of the patent application, U.S. Ser. No. 09/358,972 filed Jul. 21, 1999 (as used in Example 9 below), along with a thermostable depolymerizing enzyme such as the Tne triple mutant DNA polymerase (discussed below), Bst DNA polymerase, Ath DNA polymerase, Taq DNA polymerase and Tvu DNA polymerase along with a reaction temperature of about 50° C. to about 90° C. The use of these thermostable enzymes at an above temperature can enhance the sensitivity of the method.

The invention also contemplates a first kit embodiment for determining the presence or absence of a desired nucleic acid that contains multiple repeats of a predetermined nucleic acid target sequence in a nucleic acid sample in which the nucleic acid target sequence is repeated on average preferably at least about once for every 100,000 bases in the desired nucleic acid. The present invention is also useful for nucleic acid target sequences repeated on average at least about once for every 10,000 bases in the desired nucleic acid, on average at least about once for every 3,000 bases in the desired nucleic acid, and on average at least about once for every 300 bases in the desired nucleic acid. The first kit embodiment comprises a package containing at least one nucleic acid probe that is (i) about 10 to about 50 bases in length, and (ii) complementary to all or a portion of said repeated nucleic acid target sequence, and instructions for use. In a subset of the first kit embodiments, the kit contains a plurality of different nucleic acid probes. In a subset of the first kit embodiments, the nucleic acid probe has a length of about 10 to about 30 bases, and in a subset of that, the probe length is about 15 to about 25 bases.

In a preferred subset of the first kit embodiments, the nucleic acid probe includes a nucleic acid sequence AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11); GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15); AAGATGACGCAAATGATG (SEQ ID NO:16); GAAGATGACGCAAATGAT (SEQ ID NO:17) or a sequence complementary to those listed sequences. Preferably in most embodiments, as described above, the remainder of the probe has a sequence that is substantially homologous to the sequences adjoining the above-disclosed sequence in the nucleic acid of interest.

In a subset of the first kit embodiments, the nucleic acid probe includes a nucleic acid sequence of AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11) or a sequence complementary to those listed sequences.

In a subset of the first kit embodiments, the nucleic acid probe includes a nucleic acid sequence of GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15) or a sequence complementary to those listed sequences.

In a subset of the first kit embodiments, the nucleic acid probe includes a nucleic acid sequence of AAGATGACGCAAATGATG (SEQ ID NO:16); GAAGATGACGCAAATGAT (SEQ ID NO:17) or a sequence complementary to those listed sequences.

In a subset of the first kit embodiments, the kit also includes an enzyme whose activity is to release one or more nucleotides from the 3′ terminus of a hybridized nucleic acid probe.

In a subset of the first kit embodiments, the kit also includes adenosine 5′ diphosphate, pyrophosphate, and a nucleoside diphosphate kinase (NDPK).

### EXAMPLE 1

### Detection of Chromosomal DNA without Amplification

In theory, direct detection of a single copy gene in chromosomal DNA should be possible if enough DNA can be assayed. The amount of human genomic DNA needed can be calculated as follows:

$$\frac{( 1 \times {10}^{- 9} g DNA ) ( 5 \times {10}^{9} bases / genome )}{( 1 \times {10}^{3} bases specific target )} = approximately 5 mg of DNA$$

However, as the amount of DNA interrogated increases, nonspecific DNA signal from this DNA also increases. Therefore, chromosomal DNA in amounts approaching even 1 μg of DNA would produce very high background.

Increasing the copies of target DNA per chromosome is one way to overcome this limitation. Many such sequences are known. The absolute sequence of the repeated DNA in different species can vary as does the number of copies of the sequence in the genome. For example, there are estimated to be 500-1000 copies of a sequence known as the rep sequence in the E. coli chromosome. The Alu sequence is present in the haploid human chromosome in approximately 300,000 copies. The estimated amount of human chromosomal DNA needed to detect the Alu sequence is:

$$\frac{5 \times {10}^{- 3} grams DNA ( single copy gene requirement )}{3 \times {10}^{5} copies per genome} = 1.7 \times {10}^{- 8} grams ( or about 17 ng of DNA )$$

In this example, probes to two regions of the Alu sequence (oligonucleotide Alu 1 (SEQ ID NO:1) and oligonucleotide Alu 2 (SEQ ID NO:2)) were used to demonstrate that direct detection of chromosomal DNA is achievable.

The genomic DNA (4.2 μg) was digested to completion (5 hours, 37° C.) with 40 units of Sph I restriction enzyme, which leaves a 3′ overhang on the digested fragments. Either 40 ng or 80 ng of the digested genomic DNA was annealed to 1.0 μg of the interrogation probes Alu 1 and Alu 2 in separate reactions, and Alu 1 and Alu 2 in the same reaction with water added to a final volume of 20 μL. A negative control, without an interrogation probe, was also assembled. The solutions were heated at 92° C. for 3 minutes and cooled at room temperature for 15 minutes.

Twenty microliters of master mix, described below, were added to each annealing reaction and the tubes were further incubated at 37° C. for 20 minutes, then stored on ice. Four microliters of the reaction were added to 100 μL of L/L reagent (Promega F120B) in quadruplicate samples, and relative light units (rlu) measured on a Turner® TD20/20 luminometer. The rlu results are reported below.

Master Mix:

| | |
|---|---|
| 200 μL | 10X DNA Polymerase Buffer |
| 25 μL | 40 mM NaPPi |
| 25 μL | Klenow exo- |
| 10 μL | NDPK 1 U/μL |
| 20 μL | ADP 10 μM |
| 720 μL | water |

| | Rxn* 1 | Rxn 2 | Rxn 3 | Rxn 4 | average | Net | Std Dev* |
|---|---|---|---|---|---|---|---|
| No DNA | 3.372 | 3.342 | 3.306 | 3.249 | 3.317 | 0 | 0.052898 |
| alu1 only | 3.92 | 3.625 | 3.756 | 3.799 | 3.775 | 0.458 | 0.12174 |
| alu2 only | 23.18 | 25.47 | 24.58 | 25.19 | 24.61 | 21.29 | 1.020082 |
| 40ng DNA | 20.63 | 21.98 | 23.91 | 22.3 | 22.21 | 18.39 | 1.347504 |
| alu1 + | 53.12 | 57.05 | 52.52 | 36.5 | 49.80 | 46.03 | 9.089798 |
| 40ng DNA | | | | | | | |
| alu2 + | 99.23 | 91.26 | 55.9 | 85.59 | 83.00 | 58.39 | 18.90995 |
| 40ng DNA | | | | | | | |
| 80ng DNA | 38.57 | 44.34 | 42.96 | 46.33 | 43.05 | 39.73 | 3.291454 |
| alu1 + | 89.25 | 68.01 | 91.43 | 96.14 | 86.21 | 82.44 | 12.46776 |
| 80ng DNA | | | | | | | |
| alu2 + | 156.2 | 156.6 | 149.9 | 143.7 | 151.6 | 127.0 | 6.095353 |
| 80ng DNA | | | | | | | |
| alu1 + alu2 | 30.65 | 23.82 | 32.57 | 27.60 | 28.66 | 25.34 | 3.820881 |
| alu1 + alu2 + | 66.49 | 101.1 | 104.9 | 104.3 | 94.1975 | 65.81 | 18.54682 |
| 40ng DNA | | | | | | | |

*Std Dev = 1 standard deviation
Rxn = reaction

| | | |
|---|---|---|
| 5′ AGACCCCATCTCTAA 3′ | (Alu 1) | SEQ ID NO:1 |
| 5′ GCCTGGGTCACAGAGCA 3′ | (Alu 2) | SEQ ID NO:2 |

### EXAMPLE 2

### Detection of Mitochondrial DNA Specific to Various Animals

In this example, a segment of mitochondrial DNA comprising a segment of the cytochrome B gene was amplified from a variety of animals using PCR primers zooamp2 (SEQ ID NO:18) and zooamp1 (SEQ ID NO:19) (Proc. Nat'l. Acad. Sci., U.S.A., 86:6196-6200). These PCR primers were diluted in 10 mM Tris, pH 7.5, to a final concentration of 0.22 μg/μL. The genomic DNAs used were bovine (Clontech, 6850-1), chicken (Clontech, 6852-1), dog (Clontech, 6950-1) and human (Promega, G1521).

The PCR reactions were assembled to include 5 μL 10× buffer with 15 mM MgCl₂ (Promega, M188J), 1 μL dNTPs 10 mM (Promega, C144G), 2 μL primer 11590, 2 μL primer 11589, 0.5 μL Taq polymerase 5U/μL (Promega, M186E), and 38.5 μL water. To each tube was then added 1 μL (100 ng) of genomic DNA. The PCR cycling parameters were (15 seconds, 94° C.; 15 seconds, 55° C.; 30 seconds, 72° C.)×30. The size of PCR products was confirmed by running an aliquot on an agarose gel and visualizing with ethidium bromide (EtBr) staining. The PCR products were then separated from free nucleotides (Promega, A7170) and an aliquot run on an agarose gel. All samples produced a PCR product of the same size.

Each PCR DNA was then used in an assay to determine if it could be specifically identified with a source-specific probe. One microliter of interrogation probe (1 g/μL) and 17 μL water were combined with 2 μL of the appropriate PCR product and heated at 91° C. for 3 minutes, then cooled at room temperature for 15 minutes. Twenty microliters of master mix (described below) were added to each tube and each was further incubated at 37° C. for 15 minutes. Four microliters of the solutions were then added to 100 μL L/L reagent (Promega F120B), and the relative light output (rlu) measured on a Turner® TD20/20 luminometer. The rlu average values from two reactions, minus the DNA background values, along with the standard deviation values are listed below.

Master Mix:

312 μL 10× DNA pol buffer (Promega M195A)

39 μLNaPPi 40 mM (Promega E350B)

39 μLKlenow exo minus (Promega M128B)

15.6 μL NDPK 1 U/μL

31.2 μL ADP 10 μM (Sigma)

1123 μL water (Promega AA399)

| Averages from 2 reactions* | | | | | | Standard Deviations | | | | |
|---|---|---|---|---|---|---|---|---|---|---|
| | No | Human | Chicken | Cow | Dog | No | Human | Chicken | Cow | Dog |
| Probe | DNA | DNA | DNA | DNA | DNA | DNA | DNA | DNA | DNA | DNA |
| comzoo | −0.096 | 44.5 | 14.25 | 119.7 | 124.6 | 0.654 | 7.000 | 23.33 | 3.465 | 8.63 |
| huzoo1 | 1.771 | 38 | −40 | −51.85 | −63.55 | 0.137 | 38.96 | 31.74 | 6.505 | 12.52 |
| huzoo2 | −0.889 | 101.6 | −23.35 | −0.05 | −48.75 | 0.761 | 3.959 | 0.141 | 8.768 | 2.19 |
| chzoo1 | 43.07 | −30.4 | 34.05 | −2.75 | −31.15 | 7.078 | 1.909 | 6.364 | 2.687 | 8.70 |
| chzoo2 | −0.361 | 57.6 | 50.7 | 33.05 | −3.25 | 0.075 | 43.77 | 29.34 | 12.59 | 21.43 |
| cowzoo | 1.925 | 90.95 | 125.1 | 202.6 | 132.5 | 0.208 | 20.08 | 13.22 | 8.627 | 19.30 |
| dogzoo | 0.966 | | | 71.8 | 158.7 | 0.180 | | | 9.546 | 1.98 |

*Net light units are calculated by subtracting the DNA background

The data demonstrate that the primers detect the mitochondrial PCR product. Both of the human-specific probes, huzoo1 (SEQ ID NO:20) and huzoo2 (SEQ ID NO:21), were shown to be specific for human mitochondrial DNA. The common probe, comzoo (SEQ ID NO:22), detected all of the species, but was less efficient with chicken DNA. The chicken-specific probe, chzoo1 (SEQ ID NO:27), was specific for chicken mitochondrial DNA, but the other chicken-specific probe, chzoo2 (SEQ ID NO:28), detected all the species except dog. The cow-specific probe, cowzoo (SEQ ID NO:29), gave the best detection signal for cow DNA, but also detected the other species. The dog-specific probe, dogzoo (SEQ ID NO:30), was assayed only with dog and cow DNA, but detected the dog DNA better than cow DNA. A cleaner PCR product provides DNA with less background.

| | | |
|---|---|---|
| zooamp2 | 5′ AAACTGCAGCCCCTCAGAATGATATTTGTCCTCA 3′ | SEQ ID NO:18 |
| zooamp1 | 5′ AAAAAGCTTCCATCCAACATCTCAGCATGATGAAA 3′ | SEQ ID NO:19 |
| huzoo1 | 5′ CCAGACGCCTCA 3′ | SEQ ID NO:20 |
| huzoo2 | 5′ ACCTTCACGCCA 3′ | SEQ ID NO:21 |
| comzoo | 5′ TGCCGAGACGT 3′ | SEQ ID NO:22 |
| chzoo1 | 5′ GCAGACACATCC 3′ | SEQ ID NO:27 |
| chzoo2 | 5′ GGAATCTCCACG 3′ | SEQ ID NO:28 |
| cowzoo | 5′ ACATACACGCAA 3′ | SEQ ID NO:29 |
| dogzoo | 5′ ATATGCACGCAA 3′ | SEQ ID NO:30 |

### EXAMPLE 3

### Determination of the Minimum Cell Population that Can be Detected in a Complex Cell Mixture

This example was designed to illustrate that a small cell population can be detected in a background of other cells using a depolymerization detection method of nucleic acid hybrid detection. In this Example, a target enrichment step of RT-PCR is utilized.

RNA was isolated from a cell mixture that contained increasing numbers of K562 cells (ATCC CCL 243) in a constant background of 1×10⁶ NIH-3T3 cells (ATCC CRL 1658). The RNA was then used for RT-PCR of the bcr/abl translocation transcript, which is specific to the K562 cells due to chromosomal translocation. A translocation takes place in the region of the bcr gene along with involvement of a segment of the abl gene. The NIH-3T3 cells possess no translocation and are thus wild type for the bcr and abl transcripts.

NIH-3T3 cells were washed with phosphate-buffered saline and centrifuged at 2000× g for 5 minutes to pellet the cells. The supernatant was removed and the cell pellet was resuspended in SV RNA lysis buffer (Promega Corp., #Z3100) to a final concentration of 1×10⁶ cells/175 μL of lysis buffer. Similarly, K562 cells were washed in phosphate-buffered saline and centrifuged at 2000× g for 5 minutes to pellet the cells. The supernatant was removed and the cell pellet was resuspended in SV RNA lysis buffer to a final concentration of 1×10⁶ cells in 175 μL of lysis buffer. The cell lysates were passed through an 18-gauge needle to shear the genomic DNA.

The cell lysates were combined as listed below, and SV lysis buffer was added to a final volume of 350 μL. Sample 2 used a 1:1000 dilution of the K562 lysate, samples 3, 4, and 5 used a 1:100 dilution of the K562 lysate, and samples 6 through 10 used undiluted lysate.

| | Number | Volume (μL) | Number | Volume |
|---|---|---|---|---|
| Sample | NIH-3T3 | NIH-3T3 | K562 | (μL) K562 |
| 1 | 1 x 10⁶ | 175 | 0 | 0 |
| 2 | 1 x 10⁶ | 175 | 10 | 1.75 |
| 3 | 1 x 10⁶ | 175 | 100 | 1.75 |
| 4 | 1 x 10⁶ | 175 | 500 | 8.75 |
| 5 | 1 x 10⁶ | 175 | 1000 | 17.5 |
| 6 | 1 x 10⁶ | 175 | 1 x 10⁴ | 1.75 |
| 7 | 1 x 10⁶ | 175 | 5 x 10⁴ | 8.75 |
| 8 | 1 x 10⁶ | 175 | 1 x 10⁶ | 17.5 |
| 9 | 1 x 10⁶ | 175 | 5 x 10⁵ | 87.5 |
| 10 | 1 x 10⁶ | 175 | 1 x 10⁶ | 175 |

Total RNA was then isolated from the combined lysates listed above using the SV Total RNA Isolation System according to manufacturer's instructions (Promega, Z3100). The RNAs were each eluted into 100 μL water and stored at −20° C. until used.

The resulting RNA concentration of each sample was determined by UV spectroscopy of 10 μL of the appropriate sample mixed with 300 μL water. In set A, the samples were a combination of NIH-3T3 cells and K562 cells as listed above. In set B, the samples were only K562 cells in the number and volume as listed above. The spectroscopy results are listed below. Absorbance at OD260 measures the amount of DNA, the absorbance at OD280 measures the amount of protein, and 260/280 ratio determines the purity of the RNA with a high purity sample having a 260/280 ratio of about 2.0.

| | | | | concentration |
|---|---|---|---|---|
| Sample | OD260 | OD280 | 260/280 | (ng/mL) |
| 1A | 0.104 | 0.051 | 2.04 | 124 |
| 2A | 0.103 | 0.047 | 2.19 | 124 |
| 3A | 0.113 | 0.055 | 2.05 | 135 |
| 4A | 0.088 | 0.042 | 2.09 | 106 |
| 5A | 0.110 | 0.056 | 1.96 | 132 |
| 6A | 0.112 | 0.054 | 2.07 | 134 |
| 7A | 0.091 | 0.043 | 2.12 | 109 |
| 8A | 0.103 | 0.050 | 2.06 | 124 |
| 9A | 0.124 | 0.057 | 2.17 | 149 |
| 10A | 0.162 | 0.078 | 2.07 | 194 |
| 1B | 0.008 | 0.006 | 1.33 | na* |
| 2B | 0.005 | 0.004 | 1.25 | na |
| 3B | 0.002 | 0.004 | 0.50 | na |
| 4B | 0.001 | 0.003 | 0.33 | na |
| 5B | 0.001 | 0.001 | 1.00 | na |
| 6B | 0.002 | 0.002 | 1.00 | na |
| 7B | 0.002 | 0.003 | 0.67 | na |
| 8B | 0.001 | 0.000 | na | na |
| 9B | 0.037 | 0.018 | 2.05 | 44 |
| 10B | 0.067 | 0.303 | 2.23 | 80 |

*na = too low to be accurately measured.

The very low values for 1B through 8B indicate the lack of sensitivity for spectroscopy at very low nucleic acid concentrations as well known in the art.

RT-PCR reactions for bcr-abl product, which is K562 specific due to the chromosomal translocation, were set up using the RT-PCR system (Promega, A1250) according to manufacturer's instructions with 5 μL total RNA and the amplification primers 9261 and 10860 (below) to provide a 200 base pair RT-PCR product. The 10860 oligonucleotide used had three phosphorothioate linkages at the 5′ terminus. The RT-PCR cycling parameters were 48° C. 45 minutes, 95° C. 2 minutes, 40× (94° C. 30 seconds, 65° C. 1 minute, 68° C. 1 minute), 680C 7 minutes, 4° C. soak.

Five microliters of the resulting amplification products were run on a 1.5% agarose gel. The 200 bp bcr/abl band was detectable in samples 3B through 10B and 3A through 10A. Therefore, the bcr/abl translocation using the method described above, was easily detected in 100 K562 cells in a background of 1×10⁶ NIH-3T3 cells.

In a separate set of reactions, the products of the RT-PCR reaction (25 μL) were treated with 0.5 units of T7 gene 6 exonuclease for 30 minutes at 37° C. Samples were then purified using MagneSil™ magnetic silica particles.

Thus, twenty-five microliters of PCR product was combined with 1.5 mg of particles in 150 μL of binding buffer (0.4 M guanidine thiocyanate+0.08 M potassium acetate). The compositions were incubated for 2 minutes at room temperature. The particles were captured by placing the tube on a magnetic stand and the supernatant was removed. The particles were washed four times with 150 μL of 70% ethanol. The particles were resuspended in 50 μL of water and incubated for 2 minutes at room temperature. Then, 150 μL of binding buffer (no particles) was added and the tube incubated for 2 minutes at room temperature. The particles were captured and washed three times with 150 μL of 70% ethanol. The final wash was removed and the particles were permitted to air dry for 10 minutes at room temperature. The particles were then resuspended in 100 μL of water and incubated for 2 minutes at room temperature. The particles were captured and the supernatant containing the purified PCR product was transferred to a clean tube. When 5 μL of each purified RT-PCR product were used for an interrogation assay, a graph of the statistical data indicated that 10 K562 cells could be detected because the −4 sigma value of the 10-cell interrogation assay does not intersect with the +4 sigma value for the 0 cell control interrogation reaction. Thus, using this technique, 0.001% of a mixed cell population could be detected.

The following master mix was assembled:

| | |
|---|---|
| 10X buffer (Promega, M190) | 20 μL |
| 25 mM MgCl₂ | 20 μL |
| water | 51 μL |
| 40 mM NaPPi | 5 μL |
| 10 uM ADP | 2 μL |
| 5 U/μL Tne triple mutant | 1 μL |
| polymerase (1 U/reaction) | |
| 0.5 U/μL Pfu NDPK | 1 μL |
| (0.1 U/reaction) | |

The sample (5 μL) and the 11065 interrogation oligo (1 μg) were combined in water to a final volume of 20 μL. They were heated to 95° C. for 3 minutes, then incubated for 10 minutes at 60° C. Then 20 μL master mix was added and the reaction incubated for 15 minutes at 60° C. Then 100 μL of L/L were added, and the light output (average rlu) was measured on a Turner® TD 20/20 luminometer. The background RLU was subtracted and averaged results from 3 readings are listed below.

| | | |
|---|---|---|
| K562 cell | NIH-3T3 | bcr/abl Detection |

| number | cell number | Average rlu | Sigma |
|---|---|---|---|
| 0 | 1 x 10⁶ | 82.26 | 0.47 |
| 10 | 1 x 10⁶ | 162.43 | 9.39 |
| 100 | 1 x 10⁶ | 793.23 | 34.43 |
| 500 | 1 x 10⁶ | 1512.72 | 86.33 |
| 1000 | 1 x 10⁶ | 2042.83 | 116.02 |
| 10000 | 1 x 10⁶ | 2792.50 | 68.42 |

| | | |
|---|---|---|
| 9261 | 5′ GGAGCTGCAGATGCTGACCAAC 3′ | amplitication primer SEQ ID NO:23 |
| 10860 | 5′ GCTACTGGCCGCTGAAGGGC 3′ | amplification primer SEQ ID NO:24 |
| 11065 | 5′ GCTGACCATCAATAAGGAAG 3′ | interrogation primer SEQ ID NO:25 |

### EXAMPLE 4

### Detection of E. coli Repetitive Sequence without Nucleic Acid Amplification

In this Example repetitive sequence in E. coli is detected without the need for amplification of the target sequence prior to pyrophosphorylation. This target sequence is denoted as “colirep”.

Oligonucleotide 11707 (SEQ ID NO:26) is totally complementary to a segment of colirep DNA sequence. Twelve microliters of oligonucleotide 11707 solution (1mg/mL) were combined with 204 μL of water to make solution A. Another solution was prepared by combining 4 μL of 11707 (1 mg/mL) with 204 μL water and 8 μL 10 mM Tris, pH 8.0 to make solution B. The E. coli is Sigma cat#D4889, E. coli Strain B ultra pure.

Four nanograms (2 μL) E. coli DNA were combined with 18 μL solution A and with 18 μL solution B in separate tubes. Similarly, 40 ng E. coli DNA was combined with 18 μL solution A and with 18 μL solution B in separate tubes. These solutions were then incubated at 92° C. for 3 minutes and cooled at room temperature for 15 minutes. The following master mix was assembled:

| | |
|---|---|
| 10X DNA Polymerase buffer | 240 μL |
| 40 mM NaPPi | 30 μL |
| Klenow exo- (10 U/μL) | 30 μL |
| NDPK (1 U/μL) | 12 μL |
| 10 μM ADP (Sigma) | 24 μL |
| water | 864 μL |

Twenty microliters of master mix were added to each reaction and they were then incubated at 37° C. for 15 minutes. One hundred microliters of L/L Reagent were then added and the relative light output (rlu) immediately measured with a Turner® TD 20/20 luminometer. The rlu were:

| Solution | rlu-1 | rlu-2 | rlu-3 | Average |
|---|---|---|---|---|
| Tris | 2.85 | 3.562 | 3.059 | 3.157 |
| 11707 (A) | 13.69 | 12.13 | 13.67 | 13.16 |
| 11707 (B) | 7.473 | 7.234 | 6.981 | 7.259 |
| 40 ng DNA + Tris | 75.62 | 75.52 | 73.24 | 74.79 |
| 40 ng DNA + | 97.71 | 134.2 | 105.1 | 112.3 |
| 11707 (A) | | | | |
| 40 ng DNA + | 81.46 | 87.56 | 76.28 | 81.77 |
| 11707 (B) | | | | |
| 4 ng DNA + Tris | 6.719 | 8.084 | 5.882 | 6.895 |
| 4 ng DNA + | 24.50 | 25.97 | 25.17 | 25.21 |
| 11707 (A) | | | | |
| 4 ng DNA + | 15.69 | 17.22 | 16.99 | 16.63 |
| 11707 (B) | | | | |

The data reflect that oligonucleotide probe 11707 can detect E. coli DNA without amplification by a process of the invention.

Interrogation oligonucleotide: 11707 5′ AGTGACTGGGG 3′ SEQ ID NO:26

### EXAMPLE 5

### Detection of Human Repetitive Sequence without Nucleic Acid Amplification

The READIT™ System was demonstrated to be a highly sensitive means of identifying the presence of human genomic DNA in a sample without the need to amplify the DNA. The highly repetitive alu sequence was used as a target.

Human genomic DNA was digested for five hours at 37° C. with the Sph I restriction enzyme (Promega Corp., R6261) and then diluted in 10 mM Tris-Cl, pH 7.5 to a final concentration of 100 ng/μl. The 100 ng/μl DNA was then further diluted in 10 mM Tris-Cl, pH 7.5 to concentrations of 10 ng/μl, 1 ng/μl and 100 μg/μl.

Annealing reactions were set up to contain 4 μl of the various diluted DNAs, 1 μl Alu 1 probe (SEQ ID NO:1) or Alu 2 probe (SEQ ID NO:2) or Alu 3 probe (SEQ ID NO:3) or Alu 4 probe (SEQ ID NO:4) at a probe concentration of 1 μg/μl and 15 μl water. Control reactions containing 10 mM Tris-Cl pH 7.5 in the place of either the probe or the DNA were also assembled. The reactions were heated at about 92° C. for 3 minutes and then cooled at room temperature for 15 minutes. Then 20 μl of master mix was added to the reaction, mixed, and incubated at 37° C. for 15 minutes and stored on ice. Then 4 μl of this solution was added to 100 μl of luciferin/luciferase (hereinafter “L/L” Promega Corp., F180B and F120B, mixed and incubated at least 60 minutes at room temperature) in duplicate and the light output measured immediately in a Turner TD-20/20 luminometer. The relative light unit (rlu) output is listed in the tables below for Alu 1 and Alu 2 and in triplicate for Alu 2, Alu 3 and Alu 4.

The master mix contained reagents in the following ratios: 128 μl 10× DNAP buffer (Promega Corp., M195A), 16 μl of 40 mM NaPPi (Promega Corp., E350B), 16 μl Klenow exo minus 10 U/μl (Promega Corp., M218B), 6.4 μl Yeast NDPK 1 U/μl (Sigma Corp., N0379), 12.8 μl of 10 μM ADP, and 460.8 μl water.

| | |
|---|---|
| Alu 1 and Alu 2 | Relative Light Units |

| Sample | 1 | 2 |
|---|---|---|
| No DNA | 3.144 | 3.162 |
| Alu 1 alone | 3.595 | 3.393 |
| Alu 2 alone | 18.23 | 19.19 |
| Human DNA (40 ng) | 172.1 | 158.6 |
| Human DNA (4 ng) | 23.1 | 25.30 |
| Human DNA (400 pg) | 5. | 5.559 |
| pGEM vector (4 ng) | 3.848 | 3.465 |
| Human DNA (40 ng) + Alu 1 | 194.5 | 199.1 |
| Human DNA (4 ng) + Alu 1 | 23.6 | 23.64 |
| Human DNA (400 pg) + Alu 1 | 5.719 | 5.422 |
| Human DNA (40 ng) + Alu 2 | 248.9 | not done |
| Human DNA (4 ng) + Alu 2 | 45.09 | 41.20 |
| Human DNA (400 pg) + Alu 2 | 21.75 | 20.63 |
| pGEM vector (4 ng) + Alu 1 | | 3.291 |
| pGEM vector (4 ng) + Alu 2 | | 12.94 |

| Alu 2 Alu 3 and Alu 4 | Relative Light Units | | |
|---|---|---|---|
| Sample | 1 | 2 | 3 |
| No DNA | 5.015 | 5.065 | 5.035 |
| Alu 3 | 5.608 | 5.452 | 5.750 |
| Alu 4 | 17.68 | 15.29 | 12.86 |
| Alu 2 | 7.141 | 6.690 | 6.906 |
| Human DNA (40 ng) | 15.73 | 14.11 | 15.02 |
| Human DNA (4 ng) | 5.037 | 6.165 | 7.126 |
| Human DNA (40 ng) + Alu 2 | 30.02 | 26.96 | 29.56 |
| Human DNA (4 ng) + Alu 2 | 9.002 | 8.901 | 8.664 |
| Human DNA (40 ng) + Alu 3 | 47.39 | 46.30 | 44.69 |
| Human DNA (4 ng) + Alu 3 | 10.96 | 11.51 | 11.85 |
| Human DNA (40 ng) + Alu 4 | 34.80 | 37.92 | 35.97 |
| Human DNA (4 ng) + Alu 4 | 17.04 | 18.42 | 20.18 |

The data demonstrate that 40 ng of human genomic DNA can be detected using the READIT™ System with either Alu 1 or Alu 2 probes without PCR amplification of the human genomic DNA. The statistics for Alu 1 and Alu 2 probes; however, do not indicate a significant difference because of the small sample size. Probes Alu 3 and Alu 4 can detect human DNA without amplification. Alu 4 can detect 40 ng human DNA while Alu 3 can detect both 40 ng and 4 ng human DNA with statistical significance. The Alu 3 probe produced a rlu signal 100 percent higher than did the Alu 2 or Alu 4 probes.

| | |
|---|---|
| Alu 1 5′ AGACCCCATCTCTAA 3′ | (SEQ ID NO:1) |
| Alu 2 5′ GCCTGGGTGACAGAGCA 3′ | (SEQ ID NO:2) |
| Alu 3 5′ GACAGAGCAAGAC 3′ | (SEQ ID NO:3) |
| Alu 4 5′ TCTCGGCTCACTGCAA 3′ | (SEQ ID NO:4) |

### EXAMPLE 6

### Detection of Undigested Human DNA

In this example, human genomic DNA was demonstrated to be detectable using the READIT™ Technology with undigested human genomic DNA and probes Alu 3 (SEQ ID NO:3) and Alu 4 (SEQ ID NO:4) and a combination of probes Alu 3 plus Alu 4.

The annealing reactions were assembled in triplicate either ½ μl, 1 μl, or 2 μl of the probe at a concentration of 1 μg/μl as indicated in the data table below.

Four microliters of human genomic DNA template (either digested with Sph I or undigested) was added to 16 μl probe, heated at 94° C. for 3 minutes to denature the DNA and then cooled at room temperature for 15 minutes.

Then 20 μl of the master mix, assembled as described in Example 5, was added to the 20 μl annealing reaction described above to produce the READIT™ reaction mixture which was incubated at 37° C. for 15 minutes and then stored on ice. Then either 4 μl or 10 μl of the READIT™ reaction mixture was added to 100 μl L/L and the light output measured immediately using a Turner-TD 20/20 luminometer. The rlu are listed below in the table below.

| Template | Probe | Average +/− St. Dev. |
|---|---|---|
| none | none | 2.095 +/− 0.046 |
| *none | none | 4.064 +/− 0.080 |
| 40 ng digested human DNA | none | 11.89 +/− 0.742 |
| *40 ng digested human DNA | none | 29.93 +/− 1.972 |
| 40 ng uncut human DNA | none | 20.09 +/− 6.492 |
| none | Alu 3 (1 μl) | 2.654 +/− 0.068 |
| *none | Alu 3 (1 μl) | 5.911 +/− 0.231 |
| 40 ng digested human DNA | Alu 3 (1 μl) | 37.99 +/− 1.637 |
| *40 ng digested human DNA | Alu 3 (1 μl) | 111.1 +/− 3.717 |
| 40 ng uncut human DNA | Alu 3 (1 μl) | 41.53 +/− 2.870 |
| none | Alu 4 (1 μl) | 13.78 +/− 1.547 |
| 40 ng digested human DNA | Alu 4 (1 μl) | 30.26 +/− 2.530 |
| 40 ng uncut human DNA | Alu 4 (1 μl) | 38.09 +/− 3.471 |
| none | Alu 3 + 4 (1 μl ea.) | 14.29 +/− 2.461 |
| 40 ng digested human DNA | Alu 3 + 4 (1 μl ea.) | 30.34 +/− 1.056 |
| none | Alu 3 + 4 (½ μl ea) | 18.53 +/− 0.48 |
| 40 ng digested human DNA | Alu 3 + 4 (½ μl ea) | 39.33 +/− 0.295 |
| none | Alu 3 (2 μl) | 3.559 +/− 0.169 |
| 40 ng digested human DNA | Alu 3 (2 μl) | 43.08 +/− 0.693 |

*10 μl READIT ™ reaction was used instead of the 4 μl used in all other reactions

The Alu 3 probe worked better than Alu 4 probe or a combination of the 2 probes (Note that the Alu 3 and Alu 4 probes overlap). Adding twice the amount of the probe for Alu 3 yielded only a slight increase in luminescence signal. Overall the data demonstrate that undigested human genomic DNA is detectable with these probes, but that Alu 3 probe produced higher analytical output (rlu). Adding more of the READIT™ reaction to the 100 μl L/L gave a higher rlu, as expected.

### EXAMPLE 7

### Alu 3 Probe Source Specificity

The Alu 3 probe (SEQ ID NO:3) was used in a genomic DNA detection assay as described in Example 5, however multiple species were used as the source of the genomic DNA. Genomic DNA from chicken, cow, dog, mouse, pig, rabbit, rat, drosophila melanogaster were obtained from Clontech and diluted in 10 mM Tris-HCl, pH 7.5 to a concentration of 10 ng/μl. Human genomic DNA (Promega Corp., G147A) at a concentration of 10 ng/μl was also used.

Annealing reactions were assembled as described in Example 5 using 40 ng of undigested genomic DNA. The master mix and READIT™ reactions (in triplicate) were also assembled as described in Example 5. Then 4 μl of the READIT™ reactions were combined with 100 μl L/L and the rlu output measured in a Turner TD-20/20 luminometer. The average rlu +/− standard deviation are listed in the table below.

| | Average Relative Light Units | | |
|---|---|---|---|
| Template DNA | With Alu 3 probe | No Probe | Net rlu |
| No DNA | 5.124 +/− 0.338 | 3.982 +/− 0.255 | 1.142 |
| Human | 143.5 +/− 0.9 | 33.69 +/− 2.57 | 109.8 |
| Bovine | 37.19 +/− 3.57 | 11.39 +/− 1.37 | 25.79 |
| Chicken | 29.24 +/− 0.93 | 7.525 +/− 0.399 | 21.71 |
| Dog | 33.63 +/− 2.18 | 8.323 +/− 0.235 | 25.30 |
| Mouse | 32.49 +/− 3.33 | 13.14 +/− 0.15 | 19.35 |
| Rabbit | 59.42 +/− 2.68 | 48.78 +/− 3.18 | 10.64 |
| Dros. Melanogaster | 159.9 +/− 10.5 | 153.9 +/− 7.8 | 5.97 |
| Pig | 52.58 +/− 2.90 | 13.0 +/− 0.35 | 39.58 |
| Rat | 38.80 +/− 1.64 | 13.66 +/− 0.45 | 25.13 |

Although Alu 3 is intended to be a probe specific for human DNA, there is a lot of Alu sequence variability within each species. The data indicate that Alu 3 probe detects human DNA better than the other genomic DNA tested.

### EXAMPLE 8

### Comparison of Three Alu Probes

Alu 1 (SEQ ID NO:1), Alu 2 (SEQ ID NO:2), and Alu 3 (SEQ ID NO:3) probes were tested alone or in combination to determine which gave the best discrimination between human and mouse genomic DNA.

Human and mouse genomic DNA were as described in Example 7 at a concentration of 10 ng/μl. All Alu probes used were at a concentration of 1 μg/μl. Forty nanograms of undigested genomic DNA was used per reaction. The annealing mix, master mix, and READIT™ reactions (in triplicate) were assembled as previously described in Examples 5 and 6. When one probe was present in the annealing mix, 1 μg of the probe was used. When two probes were present in the annealing mix, 0.5 μg of each probe was used. Ten microliters of the READITM reactions were added to 100 μl L/L and the resulting light output was measured in a Turner TD-20/20 luminometer. The net average rlu (template and probe background subtracted) are listed in the first table below. The ratio of human to mouse net rlu are listed in the second table below.

| Template | Probe | Net Ave. rlu |
|---|---|---|
| Human | Alu 1 | 6.08 |
| Mouse | Alu 1 | 1.31 |
| Human | Alu 2 | 17.67 |
| Mouse | Alu 2 | 3.37 |
| Human | Alu 3 | 53.73 |
| Mouse | Alu 3 | 14.53 |
| Human | Alu 1 + Alu 2 | 16.00 |
| Mouse | Alu 1 + Alu 2 | 3.54 |
| Human | Alu 1 + Alu 3 | 37.05 |
| Mouse | Alu 1 + Alu 3 | 8.30 |
| Human | Alu 2 + Alu 3 | 37.10 |
| Mouse | Alu 2 + Alu 3 | 8.21 |

| Probe | net rlu human: mouse ratio |
|---|---|
| Alu 1 | 4.6 |
| Alu 2 | 5.2 |
| Alu 3 | 3.7 |
| Alu 1 + Alu 2 | 4.5 |
| Alu 1 + Alu 3 | 4.5 |
| Alu 2 + Alu 3 | 4.5 |

The Alu 2 probe demonstrated the best ratio of human:mouse signal, although the Alu 3 probe had the largest signal. The combination of Alu 2 and Alu 3 probes had a lower ratio of human:mouse signal than did the use of Alu 2 alone.

### EXAMPLE 9

### Thermostable Enzymes used to Detect Human Genomic DNA

Human genomic DNA and mouse genomic DNA were diluted in water to 20 ng/μl. One microgram of Alu 3 probe was combined with 40 or 80 ng of each genomic DNA in separate tubes and water added to a total volume of 20 μl. This annealing reaction was heated to 92° C. for five minutes to denature the DNA and the tubes were then allowed to cool at room temperature for 10 minutes to allow the probe to anneal to the genomic DNA.

The 2× master mix was assembled as follows:

| | | |
|---|---|---|
| 10X READIT ™ buffer (Promega A781) | 60 | μl |
| 20 mM MgCl2 (Promega A351H) | 60 | μl |
| 40 mM NaPPi (Promega C113) | 15 | μl |
| 10 μM ADP (Sigma A5285) | 6 | μl |
| Tne-3 Polymerase 5 U/μl | 3 | μl |
| Pfu NDPK 1 U/μl | 1.5 | μl |
| Water | 154.5 | μl |

Then 20 μl of the 2× master mix was added to each 20 μl annealing reaction to generate the READIT™ reaction, incubated at 55° C. for 15 minutes and stored on ice. Then 30 μl of the READIT™ reaction was added to 30 μl L/L and the resulting light output measured on a Turner TD-20/20 luminometer. The net light units, with the template and probe background subtracted, are listed in the following table.

| Template | Probe | Net light units |
|---|---|---|
| None | Alu 3 | 0.816 |
| 40 ng human DNA | Alu 3 | 13.97 |
| 80 ng human DNA | Alu 3 | 44.15 |
| 40 ng mouse DNA | Alu 3 | 1.506 |
| 80 ng mouse DNA | Alu 3 | 2.316 |

The Alu 3 probe yielded good human:mouse signal discrimination when used with thermostable enzymes in the READIT™ reaction described.

### EXAMPLE 10

### Various Probes Tested for Human-Specific DNA Detection

Five Alu specific probes were tested independently and in combination for the detection of human specific DNA. An annealing mix was prepared by combining 1 μl of each probe (at a concentration of 200 μM) included in the reaction was combined with 20 ng human genomic DNA (Promega Corp., G304A) and water to a final volume of 20 μl. The annealing mix was then heated at 92° C. for 5 minutes to denature the DNA and cooled at room temperature for 10 minutes to allow the probe to anneal to the genomic DNA. The 2× READIT™ master mix was assembled as described in Example 9 with the exception that 10× MOPS buffer at pH 6.75 was used in place of 10× READIT™ buffer (Promega A781). Then 20 μl of 2× READIT ^{T} master mix was added to each annealing reaction to create the READITM reaction that was then incubated at 50° C. for 30 minutes and stored on ice. Then 30 μl this reaction was added to 100 μl L/L and the light output measured in a Turner TD-20/20 luminometer. The results are listed in the table below.

| Probe | Template | Ave RLU | Std Deviation | Net* |
|---|---|---|---|---|
| Alu 2 | + | 50.04 | 6.05 | 15.73 |
| Alu 2 | − | 10.91 | 1.42 | |
| Alu 3 | + | 70.07 | 6.84 | 36.35 |
| Alu 3 | − | 10.31 | 0.96 | |
| Alu 10 | + | 63.27 | 2.47 | 30.21 |
| Alu 10 | − | 9.66 | 0.51 | |
| Alu 11 | + | 94.26 | 2.24 | 20.39 |
| Alu 11 | − | 50.47 | 2.07 | |
| Alu 12 | + | 89.56 | 21.70 | 55.52 |
| Alu 12 | − | 10.64 | 0.21 | |
| none | + | 30.21 | 3.02 | |
| none | − | 6.81 | 2.94 | |
| Alu 3 + Alu 10 | + | 46.03 | 11.44 | 18.24 |
| Alu 3 + Alu 10 | − | 4.39 | 0.09 | |
| Alu 3 + Alu 11 | + | 237.2 | 187.1 | 169.77 |
| Alu 3 + Alu 11 | − | 44.04 | 0.37 | |
| Alu 3 + Alu 12 | + | 119.55 | 13.08 | 84.28 |
| Alu 3 + Alu 12 | − | 11.88 | 0.42 | |
| Alu 3, 10, 11 & 12 | + | 174.25 | 16.90 | 96.73 |
| Alu 3, 10, 11 & 12 | − | 54.13 | 6.98 | |

*net value is rlu values of (probe & template) − (template alone) − (probe alone) + (blank)

Alu 12, of the probes used independently of other probes, was the best probe in that it produced high rlu signal with low background (about 10%). Alu 11 produced a good rlu signal, but the background was unacceptably high (53%). Alu 2 produced the lowest net signal. Alu 10 produced acceptably high signal and acceptably low background (15%).

The combination of Alu 10 and Alu 3 in the same reaction produced lower rlu signal than did either probe alone, interestingly the background values also drops. The combination of Alu 3 and Alu 11 gave the best signal, but a high standard deviation. The combination of Alu 3 & Alu 12 produced good signal and low standard deviation. The combination of all four probes (3, 10, 11 & 12) was acceptable had high background (31%).

| | | |
|---|---|---|
| Alu 8 | 5′ TCTCGGCTCACTGCAA 3′ | (SEQ ID NO:5) |
| Alu 10 | 5′ GGATTACAGGCGTGAG 3′ | (SEQ ID NO:6) |
| Alu 11 | 5′ TTTTTAGTAGAGCGGGG 3′ | (SEQ ID NO:7) |
| Alu 12 | 5′ GGCTGGAGTGCAGTGG 3′ | (SEQ ID NO:8) |

### EXAMPLE 11

### Alu 3L, Alu12S, and Alu 13 Probes

Two of the previously tested alu probes were modified in length and a new Alu probe, Alu 13 (SEQ ID NO:11) was designed. These probes were tested for their ability to detect human genomic DNA. Five nucleotides were added to the 5′ end of Alu 3 creating Alu3L (SEQ ID NO:9). Two nucleotides were removed from the 5′ end of Alu 12, creating Alu 12S (SEQ ID NO:10). The assay was performed using 5 ng human genomic DNA and duplicate annealing and reaction conditions as described in Example 10 with the exception that the DNA was chemically denatured as described in example 12.

Ten microliters of the READIT™ reaction was added to 100 μl L/L and the light output measured in a Turner TD-20/20 luminometer. The average rlu, standard deviation (SD), and net rlu value is listed in the following table.

| Probe | Template | Ave. rlu | SD | Net* |
|---|---|---|---|---|
| Alu 3 | + | 246.50 | 4.95 | 97.36 |
| Alu 3 | − | 66.85 | 3.55 | |
| Alu 3L | + | 218.55 | 4.45 | 72.30 |
| Alu 3L | − | 63.96 | 3.54 | |
| Alu 8 | + | 155.45 | 6.43 | 1.4 |
| Alu 8 | − | 71.76 | 14.82 | |
| Alu 12 | + | 326.20 | 1.41 | 156.41 |
| Alu 12 | − | 87.51 | 2.79 | |
| Alu 12S | + | 318.35 | 6.72 | 172.92 |
| Alu 12S | − | 63.14 | 3.51 | |
| Alu 13 | + | 235.60 | 3.54 | 87.55 |
| Alu 13 | − | 65.77 | 4.09 | |
| None | + | 145.25 | 0.49 | |
| None | − | 62.96 | 1.40 | |

*net value is rlu of (probe & template) − (template alone) − (probe alone) + (blank)

Alu 3L performs nearly as well in this assay as Alu 3. Alu 12S performs better than does Alu 12 and has a lower percent background (19.8%) than does Alu 12 (26.8%). Alu 13 was comparable to Alu 3.

| | | |
|---|---|---|
| Alu 3L | 5′ TGGGTGACAGAGCAAGAC 3′ | (SEQ ID NO:9) |
| Alu 12S | 5′ CTGGAGTGCAGTGG 3′ | (SEQ ID NO:10) |
| Alu 13 | 5′ CCACTGCACTCCAGCC 3′ | (SEQ ID NO:11) |

### EXAMPLE 12

### Enhanced Sensitivity from Using Multiple Probes to Human DNA

In this Example, it is shown that a combination of three probes to non-overlapping regions of a repeating Alu sequence provide a means for the detection of human DNA with enhanced sensitivity. Trace quantities of human genomic DNA (50 pg) was demonstrated to be detectable using the READIT™ Technology with undigested human genomic DNA and a combination of probes Alu 3 (SEQ ID NO:3), Alu 8 (SEQ ID NO:5) and Alu 13 (SEQ ID NO:11).

Human genomic DNA (Promega, G304A) was diluted with nanopure water to produce solutions in which 5 μl contained 50, 100, 200, 500, or 5000 picograms (pg) DNA. One hundred microliters of each genomic DNA dilution was combined with 100 μl 0.06 N NaOH and incubated at room temperature for 5 minutes to allow the DNA to denature into single strands.

Two probe mixes and a control containing no probe were assembled as described in the “PROBE MIXES” table below. The composition of the Master Mix is described in the “MASTER MIX” table below.

| PROBE MIXES | Probe Mix 1 | Probe Mix 2 | Control |
|---|---|---|---|
| Alu 3 (400 μM) | 25 μl | 40 μl | — |
| Alu 8 (400 μM) | 25 μl | 40 μl | — |
| Alu 13 (400 μM) | 25 μl | 40 μl | — |
| 200 mM MOPS, pH 6.5 | 500 μl | 500 μl | 500 μl |
| 20 mM MgCl₂ | | | |
| nanopure water | 425 μl | 380 μl | 500 μl |

| MASTER MIX | | |
|---|---|---|
| Nanopure water | 675 | μl |
| 200 mM MOPS, pH 6.5 | 30 | μl |
| 10 μM ADP | 75 | μl |
| NaPPi (Promega, C113A) | 30 | μl |
| READase ™ Kinase (Promega) | 3.75 | μl |
| READas ™ Polymerase | 15 | μl |

Ten microliters of the appropriate probe mix or the control was combined with 10 μl of the appropriate concentration denatured genomic DNA and 10 μl of master mix to create the reaction mixture. The reaction mixture tubes were mixed briefly and incubated at 55° C. for one hour. Then 25 μl of the reaction mixtures were each separately added to 50 μl L/L and the light output immediately read on a Turner TD20/20 luminometer. The resulting average relative light units (rlu) are listed in the Table below. The Net Signal is the average rlu in the presence of probes minus the average rlu in the absence of probes.

The data were statistically significant and demonstrate that the method detailed in this example with the combination of Alu 3, Alu 8, and Alu 13 probes at the two concentrations tested was able to detect down to 50 μpg genomic DNA without amplification. The data also demonstrate that there was a linear relationship between the rlu and the pg DNA detected.

PROBE MIX 1

| Genomic DNA (pg) | Ave. rlu | Std Dev. | % CV | Net Signal |
|---|---|---|---|---|
| Minus probes | | | | |
| 0 | 10.51 | 0.75 | 7.16 | |
| 50 | 11.06 | 0.17 | 1.50 | |
| 100 | 11.07 | 0.71 | 6.38 | |
| 200 | 13.11 | 0.49 | 3.74 | |
| 500 | 11.95 | 0.23 | 1.88 | |
| 5000 | 19.02 | 0.55 | 2.91 | |
| Plus probes | | | | |
| 0 | 11.44 | 1.37 | 12.01 | 0.93 |
| 50 | 15.72 | 0.46 | 2.91 | 4.66 |
| 100 | 19.84 | 2.28 | 11.47 | 8.77 |
| 200 | 30.49 | 4.28 | 14.38 | 17.38 |
| 500 | 57.33 | 11.73 | 20.45 | 45.38 |
| 5000 | 533.73 | 70.16 | 13.15 | 514.72 |

PROBE MIX 2

| Minus probes | | | | |
|---|---|---|---|---|
| Genomic DNA (pg) | Ave. rlu | Std. Dev. | % CV | |
| 0 | 10.57 | 0.89 | 8.38 | |
| 50 | 11.12 | 0.88 | 7.92 | |
| 100 | 9.67 | 1.83 | 18.88 | |
| 200 | 14.26 | 1.25 | 8.78 | |
| 500 | 13.18 | 0.80 | 6.07 | |
| 5000 | 19.96 | 2.41 | 12.05 | |
| Plus probes | | | | |
| Genamic DNA (pg) | Ave. rlu | Std. Dev. | % CV | Net Signal |
| 0 | 11.33 | 0.48 | 4.19 | 0.77 |
| 50 | 17.04 | 1.92 | 11.26 | 5.93 |
| 100 | 22.06 | 1.02 | 4.61 | 12.40 |
| 200 | 39.73 | 3.47 | 8.73 | 25.47 |
| 500 | 73.23 | 3.09 | 4.22 | 60.05 |
| 5000 | 729.07 | 41.38 | 5.68 | 709.10 |

### EXAMPLE 13

### E coli Repeating Sequence Probes

Four probes were designed and tested for their ability to detect E.coli rep sequence without amplification of the template nucleic acid to which they would anneal. The four probes are colirep 1 (SEQ ID NO:12), colirep 2 (SEQ ID NO:13), colirep 3 (SEQ ID NO:14) and colirep 4 (SEQ ID NO:15).

The reactions were set up in triplicate. Eighteen microliters of probe (about 1 ng/μl) was combined with either 4 ng or 40 ng of E. coli genomic DNA, incubated at 92° C. for 3 minutes, and cooled at room temperature for 15 minutes to allow the probe to anneal to the genomic DNA. To each annealing mix was added 20 μl of master mix which was prepared as described in Example 6, thereby creating the reaction mix. This reaction mix was incubated at 37° C. for 15 minutes and cooled on ice. Four microliters of the reaction mix was then combined with 100 μl L/L and the light output measured with a Turner TD-20/20 luminometer. The average relative light units (rlu), standard deviation (std), and the net light units are in the table below.

| Sample | Ave rlu | std | Net |
|---|---|---|---|
| 40 ng + colirep 1 | 46.11 | 2.803 | −28.68 |
| 40 ng + colirep 2 | 49.73 | 4.363 | −25.06 |
| 40 ng + colirep 3 | 68.45 | 7.622 | −6.34 |
| 40 ng + colirep 4 | 112.3 | 19.291 | 37.5 |
| 4 ng + colirep 1 | 9.209 | 0.217 | 2.314 |
| 4 ng + colirep 2 | 14.27 | 0.482 | 7.37 |
| 4 ng + colirep 3 | 9.235 | 1.641 | 2.340 |
| 4 ng + colirep 4 | 25.21 | 0.736 | 18.32 |

The data demonstrate that the colirep 4 probe can detect E. coli genomic DNA to amounts as low as 4 ng without amplification of the template.

| Colirep | 5′ GAATCCCCAGGAGCTTACATA 3′ | (SEQ ID NO:12) |
|---|---|---|
| 1 | | |
| Colirep | 5′ CCCAGGAGCTTACATA 3′ | (SEQ ID NO:13) |
| 2 | | |
| Colirep | 5′ GTGACCGGGGTGAGGGCGTG 3′ | (SEQ ID NO:14) |
| 3 | | |
| Colirep | 5′ AGTGACTGGGG 3′ | (SEQ ID NO:15) |
| 4 | | |

### EXAMPLE 14

### Probes for Yeast DNA Detection

Probes specific to the yeast Tyl repeat element were designed and tested for their ability to detect yeast genomic DNA in a sample without amplification.

The following master mix was assembled:

| | | |
|---|---|---|
| 500 mM MOPS, 50 mM MgCl₂, pH 6.5 | 5 | μl |
| 40 mM NaPPi | 22.5 | μl |
| Tne-3 Polymerase 5 U/μl | 9 | μl |
| Pfu NDPK 1 U/μl | 4.5 | μl |
| 10 μM ADP | 90 | μl |
| water | 320 | μl |

Annealing reactions were assembled in duplicate using 20 ng, 12 ng, and 3 ng yeast genomic DNA (Promega Corp., G310A) or 20 ng mouse genomic DNA (Clontech, 66501) in combination with 5 μl yeast Tyla or yeast Tylb probes. The probes were in 100 mM MOPS, 10 mM MgCl₂.

The annealing and reaction conditions were as described in Example 13 with the exception the DNA was chemically denatured as described in Example 12. The average rlu per nanogram genomic DNA are listed in the following table.

| Template | Probe | rlu/ng genomic DNA |
|---|---|---|
| 20 ng yeast | Tyla | 6.0925 |
| 20 ng yeast | Ty1b | 4.54 |
| 12 ng yeast | Ty1a | 4.146 |
| 12 ng yeast | Ty1b | 3.833 |
| 3 ng yeast | Ty1a | 5.46 |
| 3 ng yeast | Ty1b | 3.95 |
| 20 ng mouse | Ty1a | 0.195 |
| 20 ng mouse | Ty1b | 0.2875 |

Both yeast-specific probes were able to detect as little as 3 ng yeast genomic DNA.

Yeast Ty1a 5′ AAGATGACGCAAATGATG 3′ (SEQ ID NO:16)
Yeast Ty1b 5′ GAAGATGACGCAAATGAT 3′ (SEQ ID NO:17)

The disclosures of any patents or other publications cited in this specification are incorporated herein by reference in their entirety.

From the foregoing, it will be observed that numerous modifications and variations can be effected without departing from the true spirit and scope of the present invention. It is to be understood that no limitation with respect to the specific examples presented is intended or should be inferred. The disclosure is intended to cover by the appended claims modifications as fall within the scope of the claims.

What is claimed is:

1. A method for determining the presence or absence of a desired nucleic acid that contains multiple repeats of a predetermined nucleic acid target sequence in a nucleic acid sample that comprises the steps of:
(A) providing a treated sample that may contain said desired nucleic acid in which a plurality of said predetermined repeating nucleic acid target sequences are hybridized with a nucleic acid probe, wherein
(a) said nucleic acid target sequence is repeated in the desired nucleic acid, and
(b) said nucleic acid probe is
(i) about 10 to about 50 bases in length,
(ii) comprises a nucleic acid sequence comprising the nucleotide sequence selected from the group consisting of: AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11) GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15); AAGATGACGCAAATGATG (SEQ ID NO:16); GAAGATGACGCAAATGAT (SEQ ID NO:17) and their complements; and
(ii) is complementary to all or a portion of said repeated nucleic acid target sequence; and
(B) analyzing for the presence of hybridized nucleic acid containing said nucleic acid probe, and thereby the presence or absence of said desired nucleic acid.

2. The method according to claim 1 wherein the presence of hybridized nucleic acid containing said nucleic acid probe is analyzed for using fluorescence quenching change upon hybridization, labeled immobilization of hybrid, polymerization or depolymerization of hybridized probes.

3. The method according to claim 1 wherein a repeated sequence in said desired nucleic acid includes a plurality of predetermined repeated sequences that differ in length.

4. The method according to claim 1 wherein a repeated sequence in said desired nucleic acid includes a plurality of predetermined repeated sequences that differ in sequence.

5. The method according to claim 1 wherein a repeated sequence in said desired nucleic acid includes a plurality of predetermined repeated sequences that differ in both length and sequence.

6. The method according to claim 1 wherein said nucleic acid probe is also (iii) source-specific.

7. The method according to claim 1 wherein said nucleic acid probes include a probe comprising GACAGAGCAAGAC (SEQ ID NO:3), a probe comprising TCTCGGCTCACTGCAA (SEQ ID NO:5), and a probe comprising CCACTGCACTCCAGCC (SEQ ID NO:11).

8. A method for determining the presence or absence of a desired nucleic acid that contains multiple repeats of one or more predetermined nucleic acid target sequences in a nucleic acid sample that comprises the steps of:
(A) providing a treated sample that may contain said desired nucleic acid in which a plurality of said predetermined repeating nucleic acid target sequences are hybridized with a nucleic acid probe, wherein
(a) said nucleic acid target sequence is repeated in the desired nucleic acid, and
(b) said nucleic acid probe is
(i) about 10 to about 50 bases in length,
(ii) is complementary to all or a portion of said repeated nucleic acid target sequence; and
(iii) is source-specific; and
(B) admixing the treated sample with a depolymerizing amount of an enzyme whose activity is to release one or more nucleotides from the 3′-terminus of a hybridized nucleic acid probe to form a treated reaction mixture;
(C) maintaining the treated reaction mixture for a time period sufficient to permit the enzyme to depolymerize hybridized nucleic acid and release identifier nucleotides therefrom; and
(D) analyzing for the presence of released identifier nucleotides to obtain an analytical output, the analytical output indicating the presence or absence of said predetermined nucleic acid target sequence.

9. The method according to claim 8 wherein said nucleic acid probe is species-specific.

10. The method according to claim 8 wherein said nucleic acid probe is genus-specific.

11. The method according to claim 8 wherein said nucleic acid probe is order-specific.

12. The method according to claim 8 wherein said nucleic acid probe is specific for primate nucleic acid.

13. The method according to claim 12 wherein the primate-specific nucleic acid probe comprises the nucleotide sequence selected from the group consisting of: AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11) and their complements.

14. The method according to claim 9 wherein said nucleic acid probe is specific for E. coli.

15. The method according to claim 14 wherein the E. coli-specific nucleic acid probe comprises the nucleotide sequence selected from the group consisting of: GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15) and their complements.

16. The method according to claim 9 wherein said nucleic acid probe is specific for S. cerevisiae.

17. The method according to claim 16 wherein the yeast-specific nucleic acid probe comprises the nucleotide sequence selected from the group consisting of: AAGATGACGCAAATGATG (SEQ ID NO:16); GAAGATGACGCAAATGAT (SEQ ID NO:17) and their complements.

18. A composition comprising a nucleic acid probe that having a length of about 10 to about 50 bases in length comprising a nucleic acid sequence selected from the group consisting of: AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11); GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15); AAGATGACGCAAATGATG (SEQ ID NO:16); GAAGATGACGCAAATGAT (SEQ ID NO:17) and their complements.

19. The composition according to claim 18 comprising a nucleic acid sequence selected from the group consisting of: AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11) and their complements.

20. The composition according to claim 18 comprising a nucleic acid sequence selected from the group consisting of: GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15) and their complements.

21. The composition according to claim 18 comprising a nucleic acid sequence selected from the group consisting of: AAGATGACGCAAATGATG (SEQ ID NO:16); GAAGATGACGCAAATGAT (SEQ ID NO:17) and their complements.

22. A method for determining the presence or absence of a desired nucleic acid that contains multiple repeats of a predetermined nucleic acid target sequence in a nucleic acid sample that comprises the steps of:
(A) providing a treated sample that may contain said predetermined nucleic acid target sequence hybridized with a nucleic acid probewherein the 3′ terminal region of said nucleic acid probe is substantially complementary to all or a portion of said repeated nucleic acid target sequence;
(B) admixing the treated sample with a depolymerizing amount of an enzyme whose activity is to release one or more nucleotides from the 3′-terminus of a hybridized nucleic acid probe to form a treated reaction mixture;
(C) maintaining the treated reaction mixture for a time period sufficient to permit the enzyme to depolymerize hybridized nucleic acid and release identifier nucleotides therefrom; and
(D) analyzing the treated reaction mixture to to obtain an analytical output, the analytical output indicating the presence or absence of said predetermined nucleic acid target sequence and thus said desired nucleic acid that contains multiple repeats of the predetermined nucleic acid target sequence.

23. The method according to claim 22 wherein said identifier nucleotide is a nucleoside triphosphate.

24. The method according to claim 22 including the further steps of forming said treated sample by
(a) admixing a sample to be assayed with one or more nucleic acid probes to form a hybridization composition, wherein the 3′-terminal region of said nucleic acid probes hybridize to said predetermined nucleic acid target sequence when that sequence is present in the sample
(b) maintaining said hybridization composition for a time period sufficient to form a treated sample that may contain said predetermined nucleic acid target sequence hybridized with a nucleic acid probe sequence.

25. The method according to claim 22 wherein said nucleic acid sample is obtained from a biological sample.

26. A one-pot method for determining the presence or absence of a desired nucleic acid that contains multiple repeats of a predetermined nucleic acid target sequence in a nucleic acid sample that comprises the steps of:
(A) providing a treated sample that may contain said predetermined nucleic acid target sequence hybridized to a nucleic acid probe whose 3′-terminal region is complementary to said predetermined nucleic acid target sequence and includes an identifier nucleotide admixed with (i) a depolymerizing amount of an enzyme whose activity in the presence of pyrophosphate is to release identifier nucleotide as a nucleoside triphosphate from the hybridized nucleic acid probe, (ii) adenosine 5′ diphosphate, (iii) pyrophosphate and (iv) NDPK to form a treated reaction mixture;
(B) maintaining the treated reaction mixture at a temperature of about 25 to about 80 degrees C. for a time period sufficient to permit the enzyme to depolymerize hybridized nucleic acid probe, release an identifier nucleotide in the 3′-terminal region as a nucleoside triphosphate and to convert said nucleoside triphosphate and said adenosine 5′ diphosphate to adenosine 5′ triphosphate; and
(C) analyzing for the presence of adenosine 5′ triphosphate to obtain an analytical output, the analytical output indicating the presence or absence of at least one said nucleic acid target sequence.

27. The method according to claim 26 wherein said analytical output is obtained by luminescence spectroscopy.

28. The method according to claim 26 including the further steps of forming said treated sample by
(a) admixing a sample to be assayed with one or more nucleic acid probes to form a hybridization composition, wherein the 3′-terminal region of said nucleic acid probe (i) hybridizes with total complementarity to a nucleic acid target sequence when that sequence is present in the sample and (ii) includes an identifier nucleotide;
(b) maintaining said hybridization composition for a time period sufficient to form a treated sample that may contain said predetermined nucleic acid target sequence hybridized with a nucleic acid probe.

29. The method according to claim 26 wherein said depolymerizing enzyme maintains activity at 60-90° C.

30. The method according to claim 29 wherein said depolymerizing enzyme is selected from the group consisting of the Tne triple mutant DNA polymerase, Bst DNA polymerase, Ath DNA polymerase, Taq DNA polymerase and Tvu DNA polymerase.

31. The method according to claim 26 wherein said NDPK is that encoded by Pyrococcus furiosis.

32. A kit for determining the presence or absence of a desired nucleic acid that contains multiple repeats of a predetermined nucleic acid target sequence in a nucleic acid sample in which the nucleic acid target sequence is repeated in the desired nucleic acid that comprises at least one nucleic acid probe that is (i) about 10 to about 50 bases in length, and (ii) whose 3′ terminal region is complementary to all or a portion of said repeated nucleic acid target sequence.

33. The kit according to claim 32 that contains a plurality of different nucleic acid probes.

34. The kit according to claim 33 that contains a probe comprising GACAGAGCAAGAC (SEQ ID NO:3), a probe comprising TCTCGGCTCACTGCAA (SEQ ID NO:5), and a probe comprising CCACTGCACTCCAGCC (SEQ ID NO:11).

35. The kit according to claim 34 further comprising an enzyme whose activity is to release one or more nucleotides from the 3′ terminus of a hybridized nucleic acid probe.

36. The kit according to claim 32 wherein said nucleic acid probe comprises a nucleic acid sequence selected from the group consisting of: AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11); GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15); AAGATGACGCAAATGATG (SEQ ID NO:16); GAAGATGACGCAAATGAT (SEQ ID NO:17) and their complements.

37. The kit according to claim 36 wherein said nucleic acid probe comprises a nucleic acid sequence selected from the group consisting of: AGACCCCATCTCTAA (SEQ ID NO:1); GCCTGGGTGACAGAGCA (SEQ ID NO:2); GACAGAGCAAGAC (SEQ ID NO:3); TCTCGGCTCACTGCAA (SEQ ID NO:4); TCTCGGCTCACTGCAA (SEQ ID NO:5); GGATTACAGGCGTGAG (SEQ ID NO:6); TTTTTAGTAGAGCGGGG (SEQ ID NO:7); GGCTGGAGTGCAGTGG (SEQ ID NO:8); TGGGTGACAGAGCAAGAC (SEQ ID NO:9); CTGGAGTGCAGTGG (SEQ ID NO:10); CCACTGCACTCCAGCC (SEQ ID NO:11) and their complements.

38. The kit according to claim 36 wherein said nucleic acid probe comprises a nucleic acid sequence selected from the group consisting of: GAATCCCCAGGAGCTTACATA (SEQ ID NO:12); CCCAGGAGCTTACATA (SEQ ID NO:13); GTGACCGGGGTGAGGGCGTG (SEQ ID NO:14); AGTGACTGGGG (SEQ ID NO:15) and their complements.

39. The kit according to claim 36 wherein said nucleic acid probe comprises a nucleic acid sequence selected from the group consisting of: AAGATGACGCAAATGATG (SEQ ID NO:16); GAAGATGACGCAAATGAT (SEQ ID NO:17) and their complements.

40. The kit according to claim 36 further comprising an enzyme whose activity is to release one or more nucleotides from the 3′ terminus of a hybridized nucleic acid probe.

41. The kit according to claim 40 wherein the enzyme whose activity is to release one or more nucleotides from the 3′ terminus of a hybridized nucleic acid probe is selected from the group consisting of AMV reverse transcriptase, MMLV reverse transcriptase, DNA polymerase alpha or beta, Taq polymerase, Tth polymerase, Tne polymerase, Tne triple mutant polymerase, Tvu polymerase, Ath polymerase, E. coli DNA polymerase I, T4 DNA polymerase, Klenow fragment and Klenow exo minus.

42. The kit according to claim 40 further including adenosine 5′ diphosphate; pyrophosphate; and a nucleoside diphosphate kinase.
